(19) United States
(12) Patent Application Publication
(10) Pub. No.: US 2023/0384310 A1
(43) Pub. Date: Nov. 30, 2023

(54) METHODS OF DETECTING ANTI-AAV ANTIBODIES

(71) Applicant: Sangamo Therapeutics, Inc., Brisbane, CA (US)

(72) Inventors: Jing Hu, Brisbane, CA (US); Yanmei Lu, Brisbane, CA (US)

(21) Appl. No.: 18/029,806

(22) PCT Filed: Oct. 1, 2021
(86) PCT No.: PCT/US2021/053245

§ 371 Date: Mar. 31, 2023

Related U.S. Application Data
(60) Provisional application No. 63/086,165, filed on Oct. 1, 2020.

(51) Int. Cl. G01N 33/569; G01N 33/532

(52) U.S. Cl. CPC G01N 33/56983; G01N 33/532; G01N 2469/20; G01N 2800/52

(57) ABSTRACT

The disclosure relates to total antibody assays, including screening assays and confirmatory assays, for the detection of anti-AAV (e.g. anti-AAV6) antibodies in a subject as it relates to pre- and post-treatment total antibody levels. The disclosure also relates to methods of treating a subject with a gene therapy comprising a rAAV (e.g. rAAV6) vector.

### CROSS-REFERENCE TO RELATED APPLICATIONS

This application claims priority from U.S. Provisional Application No. 63/086,165, filed Oct. 1, 2020; the contents of which are hereby incorporated by reference in their entirety.

### BACKGROUND OF THE DISCLOSURE

Recombinant adeno-associated viruses (rAAV) are some of the most commonly used vectors for the administration of gene therapy. While there are many advantages to the use of rAAV vectors, even small amounts of anti-AAV in a subject can greatly diminish their efficacy. Because AAVs are naturally occurring viruses, there exists a non-zero baseline in the population for anti-AAV antibodies, causing variability in total antibody tools and difficulty in assessing accurate cut points for pre-treatment screening and post-treatment monitoring.

There are several serotypes of AAV used in viral gene delivery and found in both human and non-human samples. These serotypes are divergent in the amino acid sequences of their capsids (Zincarelli et al., Mol Ther., 2008; vol. 16(6): 1073-80). Accordingly, a subject who has been exposed to one AAV serotype may develop antibodies to that AAV serotype. Those anti-AAV antibodies may or may not bind to AAVs of other serotypes. Accordingly, immunogenicity analyses of anti-AAV antibody levels are critical for the enrollment of patients with no pre-existing immunity to ensure adequate treatment efficacy, and for detection of treatment-induced antibody response to assess safety.

Anti-AAV antibodies may be neutralizing antibodies or non-neutralizing antibodies. Both neutralizing and non-neutralizing antibodies can affect the efficacy and safety of AAV-based gene therapy. For example, neutralizing antibodies can inhibit uptake, affect modulation of endosomal and nuclear trafficking, influence capsid processing, and suppress gene release. Non-neutralizing antibodies can play a role in the clearance of AAV vectors, in addition to negatively affecting safety. Accordingly, it is important to have a test that detects the presence of both neutralizing and non-neutralizing antibodies. While neutralizing antibody assays are known in the art for anti-AAV (e.g. anti-AAV6) vectors, there remains a need in the art for assays to detect both neutralizing and non-neutralizing antibodies, i.e., there remains a need for a total antibody assay.

### SUMMARY OF THE DISCLOSURE

The present disclosure relates to the detection of neutralizing and non-neutralizing anti-AAV (e.g. anti-AAV6) antibodies in a subject. The present disclosure provides optimized total antibody assays, including screening assays and confirmatory assays, for more accurate detection of anti-AAV (e.g. anti-AAV6) antibodies in patient samples with higher sensitivity and reduced false positive rates. The present disclosure also relates to methods of treatment using rAAV (e.g. rAAV6) viral vectors.

A first aspect of the present disclosure provides a method of detecting anti-AAV (e.g. anti-AAV6) antibodies in a subject using a screening assay. In some embodiments, the method comprises (a) providing a test sample obtained from the test subject; (b) contacting the test sample with an AAV capsid, wherein the AAV capsid has been immobilized on a solid support; (c) contacting the test sample-contacted immobilized AAV capsid with a test secondary antibody, wherein the test secondary antibody comprises a test detectable label; and (d) detecting the test detectable label; wherein if the label detected with a normalized response relative to a negative control is greater than or equal to a cut point factor, then the test subject comprises anti-AAV antibodies; and wherein if the label is detected with a normalized response relative to a negative control is less than the cut point factor, then the test subject does not comprise anti-AAV antibodies.

In some embodiments, the cut point factor is determined by a method comprising: (1) providing reference samples obtained from a plurality of reference subjects, wherein each of the reference subjects was negative for a neutralizing anti-AAV antibody assay (i.e. prescreened negative); (2) contacting each reference sample with an AAV capsid, wherein the AAV capsid has been immobilized on a solid support; (3) contacting each reference sample-contacted immobilized AAV capsid with a reference secondary antibody, wherein the reference secondary antibody comprises a reference detectable label; (4) detecting the reference detectable label for each reference sample; (5) removing outlier reference samples; and (6) using the outlier-removed reference samples to determine the cut point factor. In some embodiments, the outlier reference samples are removed using the criteria [Q1−1.5*(Q3−Q1),Q3+1.5*(Q3−Q1)]. In some embodiments, the cut point factor is based on a parametric method with a 95% confidence interval. In some embodiments, the cut point factor is based on a robust parametric method with a 95% confidence interval. In some embodiments, the cut point factor is based on a non-parametric method.

In some embodiments, the anti-AAV antibodies are anti-AAV6 antibodies and the AAV capsid is an AAV6 capsid.

In some embodiments, the test sample comprises a fluid selected from the group consisting of serum, plasma, saliva, cerebral spinal fluid, and ocular fluid. In some embodiments, each reference sample comprises a fluid selected from the group consisting of serum, plasma, saliva, cerebral spinal fluid, and ocular fluid.

In some embodiments, the method further comprises the step of contacting the immobilized AAV capsid with a wash buffer after step (b) and before step (c). In some embodiments, the method further comprises the step of contacting the sample-contacted immobilized AAV capsid with a wash buffer after step (c) and before step (d).

In some embodiments, the test detectable label and the reference detectable label are independently selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof

In some embodiments, the test subject is a human. In some embodiments, an AAV vector was administered to the test subject before the sample was obtained from the subject.

In some embodiments, the method comprises (a) providing a sample obtained from the subject; (b) contacting the sample with an AAV (e.g. AAV6) capsid, wherein the AAV (e.g. AAV6) capsid has been immobilized on a solid support; (c) contacting the sample-contacted immobilized AAV (e.g. AAV6) capsid with a secondary antibody, wherein the secondary antibody comprises a detectable label; and (d) detecting the label; wherein if the label detected with a normalized response relative to a negative control is1.5 or greater, then the subject comprises anti-AAV (e.g. anti-AAV6) antibodies; and wherein if the label detected with a normalized response relative to a negative control is less than 1.5, then the subject does not comprise anti-AAV (e.g. anti-AAV6) antibodies.

In some embodiments, if the normalized response relative to the negative control is 1.6 or greater, then the subject comprises anti-AAV (e.g. anti-AAV6) antibodies; and wherein if the normalized response relative to the negative control is less than 1.6, then the subject does not comprise anti-AAV (e.g. anti-AAV6) antibodies. In some embodiments, if the normalized response relative to the negative control is 1.7 or greater, then the subject comprises anti-AAV (e.g. anti-AAV6) antibodies; and wherein if the normalized response relative to the negative control is less than 1.7, then the subject does not comprise anti-AAV (e.g. anti-AAV6) antibodies. In some embodiments, if the normalized response relative to the negative control is 1.73 or greater, then the subject comprises anti-AAV (e.g. anti-AAV6) antibodies; and wherein if the normalized response relative to the negative control is less than 1.73, then the subject does not comprise anti-AAV (e.g. anti-AAV6) antibodies. In some embodiments, if the normalized response relative to the negative control is 1.75 or greater, then the subject comprises anti-AAV (e.g. anti-AAV6) antibodies; and wherein if the normalized response relative to the negative control less is than 1.75, then the subject does not comprise anti-AAV (e.g. anti-AAV6) antibodies.

In some embodiments, the sample comprises a fluid selected from the group consisting of serum, plasma, saliva, cerebral spinal fluid, and ocular fluids.

In some embodiments, the method further comprises the step of contacting the immobilized AAV (e.g. AAV6) capsid with a wash buffer after step (b) and before step (c). In some embodiments, the method further comprises the step of contacting the sample-contacted immobilized AAV (e.g. AAV6) capsid with a wash buffer after step (c) and before step (d). The wash buffer may comprise 1× phosphate buffered saline, 0.05% Tween 20.

In some embodiments, the detectable label is selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the detectable label is a colorimetric label. In some embodiments, the detectable label is horseradish peroxidase.

In some embodiments, the solid support is a well of a culture plate.

In some embodiments, the subject is a mammal. In some embodiments, the subject is a human or a non-human primate. In some embodiments, the subject is a human. In some embodiments, the secondary antibody is an anti-human IgG antibody. In some embodiments, an AAV (e.g. AAV6) vector was administered to the subject before the sample was acquired from the subject.

In some embodiments, the negative control is a prescreened negative sample. In some embodiments, the negative control was negative in a neutralizing antibody assay.

A second aspect of the present disclosure provides a method of detecting anti-AAV (e.g. anti-AAV6) antibodies in a subject using a confirmatory assay.

In some embodiments, the method comprises (a) providing a first test sample and a second test sample obtained from the test subject; (b) contacting the second test sample with a test soluble AAV capsid; (c) contacting the first test sample with a first test immobilized AAV capsid, wherein the first test immobilized AAV capsid has been immobilized on a first test solid support; (d) contacting the mixture of the second test sample and the test soluble AAV capsid with a second test immobilized AAV capsid, wherein the second test immobilized AAV capsid has been immobilized on a second test solid support; (e) contacting the first test sample-contacted first test immobilized AAV capsid with a first test secondary antibody, wherein the first test secondary antibody comprises a first test detectable label; (f) contacting the mixture-contacted second test immobilized AAV capsid with a second test secondary antibody, wherein the second test secondary antibody comprises a second test detectable label, wherein the second test secondary antibody is the same as the first test secondary antibody and the first test detectable label is the same as the second test detectable label; and (g) detecting the first and second test detectable labels; wherein if the amount of second label detected is reduced by a cut point percentage or more compared to the amount of first label detected, then the subject comprises anti-AAV antibodies; and wherein if the amount of second label detected is reduced by less than the cut point compared to the amount of first label detected, then the subject does not comprise anti-AAV antibodies.

In some embodiments, the cut point is determined by a method comprising: (1) providing first and second reference samples obtained from a plurality of reference subjects, wherein each of the reference subjects was negative for a neutralizing anti-AAV antibody assay; (2) contacting, for each reference subject, the second reference sample with a reference soluble AAV capsid; (3) contacting, for each reference subject, the first reference sample with a first reference immobilized AAV capsid, wherein the first reference immobilized AAV capsid has been immobilized on a first reference solid support; (4) contacting, for each reference subject, the mixture of the second reference sample and the reference soluble AAV capsid with a second reference immobilized AAV capsid, wherein the second reference immobilized AAV capsid has been immobilized on a second reference solid support; (5) contacting, for each reference subject, the first reference sample-contacted first immobilized AAV capsid with a first secondary antibody, wherein the first secondary antibody comprises a first reference detectable label; (6) contacting, for each reference subject, the mixture-contacted second immobilized AAV capsid with a second secondary antibody, wherein the second secondary antibody comprises a second reference detectable label, wherein the second secondary antibody is the same as the first secondary antibody and the first reference detectable label is the same as the second reference detectable label; (7) detecting, for each reference subject, the first and second reference detectable labels; (8) removing outlier reference samples; and (9) using the outlier-removed reference samples to determine the cut point percentage.

In some embodiments, the outlier reference samples are removed using the criteria [Q1−1.5*(Q3−Q1),Q3+1.5*(Q3−Q1)]. In some embodiments, the cut point percentage is based on a parametric method with a 99% confidence interval. In some embodiments, the cut point percentage is based on a robust parametric method with a 99% confidence interval. In some embodiments, the cut point percentage is based on a non-parametric method.

In some embodiments, the anti-AAV antibodies are anti-AAV6 antibodies, the soluble AAV capsid is a soluble AAV6 capsid, the first immobilized AAV capsid is a first immobilized AAV6 capsid, and the second immobilized AAV capsid is a second immobilized AAV6 capsid.

In some embodiments, the first and second test sample each comprises a fluid selected from the group consisting of serum, plasma, saliva, cerebral spinal fluid, and ocular fluid. In some embodiments, each first and second reference sample comprises a fluid selected from the group consisting of serum, plasma, saliva, cerebral spinal fluid, and ocular fluid.

In some embodiments, the method further comprises the step of contacting the first immobilized AAV6 capsid with a wash buffer after step (c) and before step (e). In some embodiments, the method further comprises the step of contacting the second immobilized

AAV6 capsid with a wash buffer after step (d) and before step (f). In some embodiments, the method further comprises the step of contacting the sample-contacted first immobilized AAV6 capsid with a wash buffer after step (e) and before step (g). In some embodiments, the method further comprises the step of contacting the mixture-contacted second immobilized AAV6 capsid with a wash buffer after step (f) and before step (g).

In some embodiments, the first and second test detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the first and second reference detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.

In some embodiments, the subject is a human. In some embodiments, an AAV vector was administered to the subject before the sample was obtained from the sample.

In some embodiments, the soluble AAV capsid is at a concentration of 7.5E10 to 15E10 cp/mL.

In some embodiments, the method comprises (a) providing a first sample and a second sample obtained from the subject; (b) contacting the second sample with a soluble AAV (e.g. AAV6) capsid; (c) contacting the first sample with a first immobilized AAV (e.g. AAV6) capsid, wherein the first immobilized AAV (e.g. AAV6) capsid has been immobilized on a first solid support; (d) contacting the mixture of the second sample and the soluble AAV (e.g. AAV6) capsid with a second immobilized AAV (e.g. AAV6) capsid, wherein the second immobilized AAV (e.g. AAV6) capsid has been immobilized on a second solid support; (e) contacting the first sample-contacted first immobilized AAV (e.g. AAV6) capsid with a first secondary antibody, wherein the first secondary antibody comprises a first detectable label; (f) contacting the mixture-contacted second immobilized AAV (e.g. AAV6) capsid with a second secondary antibody, wherein the second secondary antibody comprises a second detectable label, wherein the second secondary antibody is the same as the first secondary antibody and the second detectable label is the same as the second detectable label; and (g) detecting the first and second labels; wherein if the amount of second label detected is reduced by 20% or more compared to the amount of first label detected, then the subject comprises anti-AAV (e.g. anti-20 AAV6) antibodies; and wherein if the amount of second label detected is reduced by less than 20% compared to the amount of first label detected, then the subject does not comprise anti-AAV (e.g. anti-AAV6) antibodies.

In some embodiments, if the amount of second label detected is reduced by 22% or more compared to the amount of first label detected, then the subject comprises anti-AAV (e.g. anti-AAV6) antibodies; and wherein if the amount of second label detected is reduced by less than 22% compared to the amount of first label detected, then the subject does not comprise anti-AAV (e.g. anti-AAV6) antibodies. In some embodiments, if the amount of second label detected is reduced by 24% or more compared to the amount of first label detected, then the subject comprises anti-AAV (e.g. anti-AAV6) antibodies; and wherein if the amount of second label detected is reduced by less than 24% compared to the amount of first label detected, then the subject does not comprise anti-AAV (e.g. anti-AAV6) antibodies. In some embodiments, if the amount of second label detected is reduced by 24.1% or more compared to the amount of first label detected, then the subject comprises anti-AAV (e.g. anti-AAV6) antibodies; and wherein if the amount of second label detected is reduced by less than 24.1% compared to the amount of first label detected, then the subject does not comprise anti-AAV (e.g. anti-AAV6) antibodies. In some embodiments, if the amount of second label detected is reduced by 24.5% or more compared to the amount of first label detected, then the subject comprises anti-AAV (e.g. anti-AAV6) antibodies; and wherein if the amount of second label detected is reduced by less than 24.5% compared to the amount of first label detected, then the subject does not comprise anti-AAV (e.g. anti-AAV6) antibodies.

In some embodiments, the first and second sample each comprises a fluid selected from the group consisting of serum, plasma, saliva, cerebral spinal fluid, and ocular fluid.

In some embodiments, the method further comprises the step of contacting the first immobilized AAV (e.g. AAV6) capsid with a wash buffer after step (c) and before step (e). In some embodiments, the method further comprises the step of contacting the second immobilized AAV (e.g. AAV6) capsid with a wash buffer after step (d) and before step (f). In some embodiments, the method further comprises the step of contacting the sample-contacted first immobilized AAV (e.g. AAV6) capsid with a wash buffer after step (e) and before step (g). In some embodiments, the method further comprises the step of contacting the mixture-contacted second immobilized AAV (e.g. AAV6) capsid with a wash buffer after step (f) and before step (g). In some embodiments, the wash buffer comprises of 1× phosphate buffered saline, 0.05% tween.

In some embodiments, the first and second detectable label are each selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the first and second detectable labels are each a colorimetric label. In some embodiments, the first and second detectable labels are each horseradish peroxidase.

In some embodiments, the first and second solid support are each independently a well of a culture plate.

In some embodiments, the subject is a mammal. In some embodiments, the subject is a human or a non-human primate. In some embodiments, the subject is a human. In some embodiments, the secondary antibody is an anti-human IgG antibody. In some embodiments, an AAV (e.g. AAV6) vector was administered to the subject before the first and second sample were acquired from the subject.

In some embodiments, the soluble AAV (e.g. AAV6) capsid is at a concentration of 7.5E10-1.5E11 cp/mL.

A third aspect of the present disclosure provides a method of treating a subject (e.g. a test subject) in need of gene therapy. In some embodiments, the method comprises providing the subject (e.g. a test subject) and administering a recombinant adeno-associated virus (rAAV, e.g. rAAV6) vector to the subject (e.g. a test subject) only if the subject does not comprise anti-AAV (e.g. anti-AAV6) antibodies. In some embodiments, any anti-AAV (e.g. anti-AAV6) antibodies in the subject (e.g. a test subject) are detected by any of the assay methods disclosed herein.

In some embodiments, the subject (e.g. a test subject) is a mammal. In some embodiments, the subject (e.g. a test subject) is a human or a non-human primate. In some embodiments, the subject (e.g. a test subject) is a human. In some embodiments, the secondary antibody is an anti-human IgG antibody. In some embodiments, the subject (e.g. a test subject) has not previously been administered an AAV (e.g. AAV6) vector.

In some embodiments, the subject (e.g. a test subject) is suffering from a genetic disorder. In some embodiments, the subject (e.g. a test subject) is suffering from a neurological disorder. In some embodiments, the disorder is selected from the group consisting of hemophilia A and B, Fabry disease, sickle cell disease, beta thalassemia, mucopolysaccharidosis type I and II, phenylketonuria, glycogen storage disease type 1a, GLUT1 deficiency syndrome, and HIV/AIDS. In some embodiments, the disorder is Fabry disease.

### BRIEF DESCRIPTION OF DRAWINGS

FIGS. 1A-1C provide graphical depictions of exemplary AAV antibody detection assays. Panel A provide the graphical depiction of an exemplary AAV neutralizing activity assay using AAV vectors comprising a luciferase reporter gene. Panel B provides a graphical depiction of an exemplary AAV screening assay using immobilized AAV capsids. Panel C provides a graphical depiction of an exemplary AAV confirmatory assay using both immobilized and soluble AAV capsids.

FIG. 2 provides the results of an AAV6 screening assay using samples that were prescreened negative for an AAV6 neutralizing activity assay. The serum samples (n=38) were graphed by optical density (OD). Negative controls used to calculate normalized response were pooled from a subset of these samples. The same prescreening method were performed on other individuals to select donor samples for cut point determination.

FIGS. 3A and 3B provide schematics of the method for determining the cut point. Panel A provides a diagram of a box plot used for outlier removals. Quartile (Q) 1 and Q3 were used to calculate outliers using the calculations provided. Panel B provides a graph of the normalized response to use to determine the cut point for anti-drug antibodies (ADA) in total antibody assay (screening assay (false positive rate (FPR) 5%)) and confirmatory assay (FPR 1%)). The parametric calculations for determining cut points were: Mean+1.645×SD (5% FPR) or Mean+2.33×SD (1%FPR). SD: standard deviation.

FIGS. 4A and 4B provide box plot diagrams for 100 serum samples without prescreening. Panel A provides the cut point factors with one, two, or three rounds of outlier removal. Following 3 rounds of outlier removals, 70 samples were plotted for in the final cut point factor calculation, which determined a final cut point factor of 2.61 for the screening assay. The outlier removal strategy affected the cut point factor calculation. Panel B provides the box plot diagram of data from 100 random serum samples in a confirmatory assay. No outliers were identified. After removing the outliers identified in the screening assay, the cut point percentage was determined to be 43.1% inhibition.

FIGS. 5A and 5B provide box plot and distribution of data from serum samples pre-screened by neutralizing activity assay. Panel A provides the box plot calculation and log-transformed normalized response plot of the screening assay using 211 serum samples that were pre-screened using a neutralizing activity assay and determined to be negative for anti-AAV6 neutralizing antibodies (“pre-screened negative serum samples”). The cut point factor for the screening assay was calculated as 1.73. Panel B provides the box plot and plot of a confirmatory assay using 211 pre-screened negative serum samples. The cut point percentage for the confirmatory assay was calculated to be 24.5% inhibition.

FIGS. 6A and 6B provide the results of positive control screening. Panel A provides the results of a screening assay using serum samples from 63 random human subjects graphed by optical density (OD). The arrows identify the 5 samples with the highest signal. Panel B provides a screening assay using a titration of the 5 samples with the highest positive signals. The sample with the highest OD at the lowest concentration (i.e., highest dilution), PC-2, identified by the arrow, was selected to generate the positive control.

FIGS. 7A and 7B provide sensitivity determinations for the total antibody assays. Panel A provides sensitivity determinations for the screening assay using titrations with either IgG antibodies (top panel) or anti-AAV6 specific antibodies isolated from serum sample PC-2 using Protein A/G and AAV6-coupled magnetic beads, respectively. The assay sensitivity using all IgG antibodies was determined to be 15.4 μg/mL, and the assay sensitivity using anti-AAV6 specific antibodies was determined to be 0.241 μg/mL. Panel B provides sensitivity determinations for the confirmatory assay using titrations with either IgG antibodies (top panel) or anti-AAV6 specific antibodies isolated from serum sample PC-2 using Protein A/G and AAV6-coupled magnetic beads, respectively. The assay sensitivity using all IgG antibodies was determined to be 5.85 μg/mL, and the assay sensitivity using anti-AAV6 specific antibodies was determined to be 0.0734 μg/mL.

FIGS. 8A-8C provide plots of a screening assay vs a neutralizing antibody assay. Panel A provides a comparison between the total antibody (TAb) screening assay using the cut point factor derived from random serum samples (CPF=2.6) and the neutralizing antibody (NAb) assay (CPF=0.3) for the detection of anti-AAV6 antibodies in a panel of 100 random healthy subjects. The oval indicates false negatives in the TAb screening assay. Panel B provides a comparison between the total antibody (TAb) screening assay using the cut point factor derived from pre-screened negative samples (CPF=1.75) and the neutralizing antibody (NAb) assay (CPF=0.3) for the detection of anti-AAV6 antibodies in a panel of 100 random healthy subjects. No false negative results were identified. Panel C provides a comparison between the confirmatory assay (Tab % inhibition) using the cut point percentage derived from pre-screened negative samples (CPP=24.5% inhibition) and the neutralizing antibody (NAb) assay (CPF=0.3; i.e., 30% RLU) for the detection of anti-AAV6 antibodies in a panel of 100 random healthy subjects. No false negative results were identified.

FIGS. 9A and 9B show the selectivity of the screening assay and the confirmatory assay. Panel A confirms the selectivity of the screening assay. Ten pre-screened negative samples were spiked with 20 μg/mL and 40 μg/mL of total IgG positive control. The screening assay did not detect anti-AAV6 antibodies in the pre-screened negative samples but did detect anti-AAV6 antibodies in every spiked sample. NR=normalized response. Panel B confirms the selectivity of the confirmatory assay. Ten pre-screened negative samples were spiked with 20 μg/mL and 40 μg/mL of total IgG positive control. The confirmatory assay detected anti-AAV6 antibodies in every spiked sample.

### DETAILED DESCRIPTION OF THE DISCLOSURE

### 1. General Techniques

Unless otherwise defined herein, scientific and technical terms used in this application shall have the meanings that are commonly understood by those of ordinary skill in the art. Generally, nomenclature used in connection with, and the laboratory procedures techniques performed in pharmacology, cell and tissue culture, analytical chemistry, biochemistry, synthetic organic chemistry, medicinal and pharmaceutical chemistry, molecular biology, cell and cancer biology, neurobiology, neurochemistry, virology, immunology, microbiology, genetics and protein and nucleic acid chemistry, described herein, are those well-known and commonly used in the art. Standard techniques are used for chemical syntheses and chemical analyses. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein.

Throughout this specification and embodiments, the word “comprise,” or variations such as “comprises” or “comprising,” will be understood to imply the inclusion of a stated integer or group of integers, but not the exclusion of any other integer or group of integers. The term “including” is used to mean “including but not limited to.” “Including” and “including but not limited to” are used interchangeably.

It is understood that wherever embodiments are described herein with the language “comprising,” otherwise analogous embodiments described in terms of “consisting of” and/or “consisting essentially of” are also provided.

Any example(s) following the term “e.g.” or “for example” is not meant to be exhaustive or limiting.

Unless otherwise required by context, singular terms shall include pluralities and plural terms shall include the singular.

The articles “a,” “an” and “the” are used herein to refer to one or to more than one (i.e., to at least one) of the grammatical object of the article. By way of example, “an element” means one element or more than one element. As used herein, the terms “about” and “approximately” modifying the quantity of an ingredient, parameter, calculation, or measurement in the compositions of the disclosure or employed in the methods of the disclosure refers to a variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10% of a given value or range, more typically still within 5% of a given value or range. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the invention are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the embodiments of the invention.

Exemplary methods and materials are described herein, although methods and materials similar or equivalent to those described herein can also be used in the practice or testing of the present disclosure. The materials, methods, and examples are illustrative only and not intended to be limiting.

### 2. Definitions

Unless otherwise defined herein, all scientific and technical terms used in this application are commonly understood by those with general knowledge of the previous art that this disclosure involves. Generally, these terms will be used in connection with techniques associated with molecular biology, immunology, cell culture, and analytical biochemistry and are well-known and commonly used in the art. Therefore, the specifications and definitions provided are done to reduce any conflict of terminology.

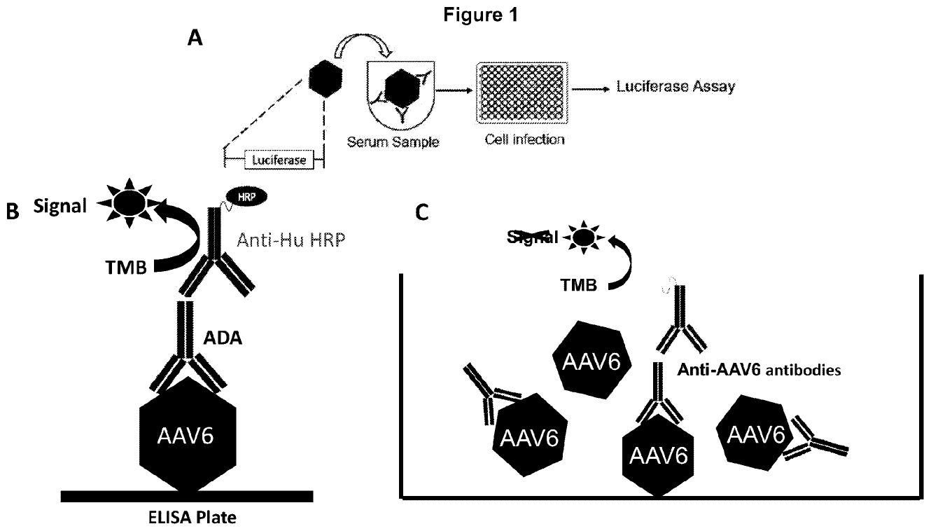

The term “confirmatory assay,” as used herein, refers to a competition assay. See, e.g., FIG. 1C. For example, a sample obtained from a patient can be incubated with a soluble AAV (e.g. AAV6) capsid (or portion thereof) before the sample contacts an immobilized AAV (e.g. AAV6) capsid (or portion thereof). The soluble molecule competes with the immobilized molecule for binding to any anti-AAV (e.g. anti-AAV6) antibodies in the sample. Such competition reduces the number of captured anti-AAV (e.g. anti-AAV6) antibodies in the assay and, therefore, the number of secondary antibodies bound, via captured anti-AAV (e.g. anti-AAV6) antibodies, to the immobilized molecule and reduces the overall signal from the secondary antibodies. Such reduction in signal is referred to herein as “percent inhibition.”

The terms “cut point factor” and “CPF,” as used herein, refer to the threshold signal in a total antibody screening assay or a screening assay for the detection of anti-AAV (e.g. anti-AAV6) antibodies in a sample. A signal at the threshold signal or greater indicates that antibodies have been detected. A signal below the threshold signal indicates that antibodies have not been detected. In some embodiments, the signal is a normalized value, e.g. normalized to a negative control. In such embodiments, a “cut point” signal can be calculated for a particular assay. For example, in an assay measuring optical density (OD), the “cut point OD” can be determined by multiplying the CPF by the mean OD signal from the negative controls. The CPF may be determined using a parametric method, a robust parametric method, or a non-parametric method. In some embodiments, the CPF is determined using a parametric method. In some embodiments, the CPF is determined using a robust parametric method. In some embodiments, the CPF is determined using a non-parametric method. In some embodiments, the CPF is determined from samples obtained from a population reference subjects, each of whom was negative for a neutralizing anti-AAV antibody assay.

The terms “cut point percentage” and “CPP,” as used herein, refer threshold percent inhibition in a confirmatory assay for the detection of anti-AAV (e.g. anti-AAV6) antibodies in a sample. A percent inhibition at the threshold signal or greater indicates that antibodies have been detected. A percent inhibition below the threshold signal indicates that antibodies have not been detected. The CPP may be determined using a parametric method, a robust parametric method, or a non-parametric method. In some embodiments, the CPP is determined using a parametric method. In some embodiments, the CPP is determined using a robust parametric method. In some embodiments, the CPP is determined using a non-parametric method. In some embodiments, the CPP is determined from samples obtained from a population reference subjects, each of whom was negative for a neutralizing anti-AAV antibody assay.

The terms “detection antibody” and “secondary antibody” are used interchangeably herein, and refer to an antibody that binds an anti-AAV (e.g. anti-AAV6) antibody and allow detection of that antibody in a sample. Accordingly, the detection antibody is typically conjugated to a detectable label, such as horseradish peroxidase. In some embodiments, the detection antibody is an anti-IgG antibody. In some embodiments, the detection antibody specifically binds antibodies from the species of the subject from which the sample was obtained. For example, anti-human IgG antibodies may be used detection antibodies for binding anti-AAV (e.g. anti-AAV6) antibodies in a sample obtained from a human subject.

The term “non-parametric method,” as used herein, refers to a method of determining a CPF or a CPP. A non-parametric method is used for populations with a Shapiro-Wilk P<0.05 and a skewness>1 or <−1. A CPF for a screening assay based on the non-parametric method is the 95^{th} percentile, and a CPP for a confirmatory assay based on the non-parametric method is the 99^{th} percentile.

The terms “normalized response” and “NR,” as used herein, refer to the ratio of a signal from a sample to the signal from a standard used in the assay. In some embodiments, the standard is a negative control. For example, the signal from each sample may be divided by the mean signal from the negative controls, as defined by the assay. In some embodiments, the normalized response for a screening assay is calculated as follows:

$$Normalized response = \frac{Mean OD of sample replicates}{Mean OD of negative control replicates}$$

The terms “optical density” and “OD,” as used herein, refer to a measure of concentration and are defined by the degree of which the sample absorbs or reflects light. Accordingly, the optical density (OD) of the sample may be compared to a standard curve to determine the concentration of a given component. The standard curve may be generated using the serial dilution, e.g., of a positive control. In some embodiments, the signal from a detectable label (e.g. a colorimetric label, such as horseradish peroxidase) is measure by optical density.

The term “parametric method,” as used herein, refers to a method of determining a CPF or a CPP. A parametric method is used for populations with a Shapiro-Wilk P>0.05. In some embodiments, the CPF or CPP is based on a parametric method with a 95% confidence interval and is equal to the mean+(1.645×SD). In some embodiments, the CPF or a CPP is based on a parametric method with a 99% confidence interval and is equal to the mean+(2.33×SD). SD: standard deviation.

The term “percent inhibition” as used herein, refers to the reduction in signal in the sample comprising soluble AAV (e.g. AAV6) capsids in a confirmatory assay. The percent inhibition may be calculated by the following formula:

$$\% inhibition = ( 1 - \frac{soluble rAAV6 inhibited sample OD}{uninhibited samples OD} )$$

The response can be normalized or raw. In some embodiments, the percent inhibition can be expressed as a decimal. For example, a 30% inhibition may also be referred to as a 0.3 inhibition. When expressed as a decimal, the percentage sign (%) is omitted.

The term “robust parametric method,” as used herein, refers to a method of determining a CPF or a CPP. A robust parametric method is used for populations with a Shapiro-Wilk P<0.05 and a −1<skewness<1. In some embodiments, the CPF or CPP is based on a robust parametric method with a 95% confidence interval and is equal to the median+(1.645×(1.483×MAD)). MAD: median absolute deviation. In some embodiments, the CPF or CPP is based on a robust parametric method with a 99% confidence interval and is equal to the median+(2.33×SD). SD: standard deviation.

The term “sample,” as used herein, refers to any tissue or fluid obtained from a subject that can be used to detect anti-AAV (e.g. anti-AAV6) antibodies. The sample obtained from the subject can be modified prior to testing and still be considered a “sample” herein. For example, if a vial of blood is obtained from a subject, and the serum portion of the blood may be isolated from other components of the blood. In the present disclosure, such isolated serum is considered a sample obtained from the patient. Similarly, a single sample may be obtained from a patient and then divided into two or more samples. Each sample is still considered to be obtained from the subject. For example, in a confirmatory assay, a single serum sample may be obtained from a subject and divided into a first sample, which is not contacted by a soluble AAV (e.g. AAV6) capsid, and a second sample, which is contacted by a soluble AAV (e.g. AAV6) capsid. The first sample and second sample are both considered samples obtained from the subject.

The terms “screening assay” and “total antibody screening assay” are used interchangeably herein and refer to an antibody test to detect the presence of anti-AAV (e.g. anti-AAV6) antibodies. See, e.g., FIG. 1B. A screening assay typically utilizes immobilized AAV (e.g. AAV6) capsids (or portions thereof) to capture anti-AAV (e.g. anti-AAV6) antibodies in the sample. The captured antibodies can then be detected using a secondary antibody conjugated to a detectable label. For example, a screening assay may be an ELISA using a horseradish peroxidase conjugated secondary antibody. A screening assay is typical interpreted according to a cut point factor.

The terms “subject,” “patient” and “individual” are used interchangeably herein and refer to a human or a non-human animal from whom a sample is taken from for anti-AAV (e.g. anti-AAV6) antibody detection, use as a negative control, and/or positive control generation. In some embodiments, the subject is in need of gene therapy. In some embodiments, the subject is in need of treatment with an AAV (e.g. AAV6) vector. In some embodiments, the subject has received treatment with an AAV (e.g. AAV6) vector. In some embodiments, the subject has not received treatment with an AAV (e.g. AAV6) vector. These terms include mammals, such as humans, non-human primates (e.g., monkeys), and laboratory animals (e.g., mice, rats, rabbits, guinea pigs, dogs, pigs, etc.). In some embodiments, the subject is a human.

The term “total antibody assay,” as used herein, refers to a test used to detect the presence of anti-AAV (e.g. anti-AAV6) antibodies in a sample, regardless of whether the anti-AAV (e.g. anti-AAV6) antibodies are neutralizing antibodies or non-neutralizing antibodies. The screening assays and confirmatory assays disclosed herein are total antibody assays.

### 3. Detection of Anti-AAV (e.g. anti-AAV6) Antibodies Using A Screening Assay

A first aspect of the present disclosure provides a method of detecting anti-AAV (e.g. anti-AAV6) antibodies in a subject using a screening assay. In some embodiments, the method comprises (a) providing a test sample obtained from the test subject; (b) contacting the test sample with an AAV capsid, wherein the AAV capsid has been immobilized on a solid support; (c) contacting the test sample-contacted immobilized AAV capsid with a test secondary antibody, wherein the test secondary antibody comprises a test detectable label; and (d) detecting the test detectable label; wherein if the label detected with a normalized response relative to a negative control is greater than or equal to a cut point factor, then the test subject comprises anti-AAV antibodies; and wherein if the label is detected with a normalized response relative to a negative control is less than the cut point factor, then the test subject does not comprise anti-AAV antibodies.

In some embodiments, the cut point factor is determined by a method comprising: (1) providing reference samples obtained from a plurality of reference subjects, wherein each of the reference subjects was negative for a neutralizing anti-AAV antibody assay (i.e. prescreened negative); (2) contacting each reference sample with an AAV capsid, wherein the AAV capsid has been immobilized on a solid support; (3) contacting each reference sample-contacted immobilized AAV capsid with a reference secondary antibody, wherein the reference secondary antibody comprises a reference detectable label; (4) detecting the reference detectable label for each reference sample; (5) removing outlier reference samples; and (6) using the outlier-removed reference samples to determine the cut point factor. In some embodiments, the outlier reference samples are removed using the criteria [Q1−1.5*(Q3−Q1),Q3+1.5*(Q3−Q1)]. In some embodiments, the use of prescreened negative subjects for determining the cut point factor results in at least one fewer round of outlier removal compared to the use of random subjects for determining the cut point factor. In some embodiments, the cut point factor is based on a parametric method with a 95% confidence interval (i.e. is equal to the mean+(1.645×SD)). In some embodiments, the cut point factor is based on a parametric method with a 99% confidence interval (i.e. is equal to the mean+(2.33×SD)). In some embodiments, the cut point factor is based on a robust parametric method with a 95% confidence interval (i.e. is equal to the median+(1.645×(1.483×MAD))). In some embodiments, the cut point factor is based on a robust parametric method with a 99% confidence interval (i.e. is equal to the median+(2.33×SD)). In some embodiments, the cut point factor is based on a non-parametric method (i.e. the 95th percentile).

In some embodiments, the anti-AAV antibodies are anti-AAV6 antibodies and the AAV capsid is an AAV6 capsid.

In some embodiments, the test sample comprises a fluid selected from the group consisting of serum, plasma, saliva, cerebral spinal fluid, and ocular fluid. In some embodiments, each reference sample comprises a fluid selected from the group consisting of serum, plasma, saliva, cerebral spinal fluid, and ocular fluid.

In some embodiments, the method further comprises the step of contacting the test immobilized AAV capsid with a wash buffer after step (b) and before step (c). In some embodiments, the method further comprises the step of contacting the sample-contacted test immobilized AAV capsid with a wash buffer after step (c) and before step (d). The wash buffer may comprise 1× phosphate buffered saline, 0.05% Tween 20. Tween 20 is also known as polysorbate 20. In some embodiments, the wash is repeated 3 times. In some embodiments, the wash is done using a plate washer. In some embodiments, the wash is done at room temperature.

In some embodiments, the method further comprises the step of contacting the reference immobilized AAV (e.g. AAV6) capsid with a wash buffer after step (2) and before step (3). In some embodiments, the method further comprises the step of contacting the sample-contacted reference immobilized AAV (e.g. AAV6) capsid with a wash buffer after step (3) and before step (4). The wash buffer may comprise 1× phosphate buffered saline, 0.05% Tween 20. Tween 20 is also known as polysorbate 20. In some embodiments, the wash is repeated 3 times. In some embodiments, the wash is done using a plate washer. In some embodiments, the wash is done at room temperature.

In some embodiments, the test detectable label and the reference detectable label are independently selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the test detectable label is a fluorescent label. In some embodiments, the test detectable label is a fluorogenic label. In some embodiments, the test detectable label is a dye In some embodiments, the test detectable label is a colorimetric label. In some embodiments, the test detectable label is a radioactive label. In some embodiments, the test detectable label is a luminescent label. In some embodiments, the test detectable label is a chemiluminescent label. In some embodiments, the test detectable label is a magnetic particle. In some embodiments, the test detectable label is a metal particle. In some embodiments, the test detectable label is a charged particle. In some embodiments, the test detectable label is an ionic solution. In some embodiments, the test detectable label is a spore. In some embodiments, the test detectable label is an enzymatic label.

In some embodiments, the reference detectable label is a fluorescent label. In some embodiments, the reference detectable label is a fluorogenic label. In some embodiments, the reference detectable label is a dye In some embodiments, the reference detectable label is a colorimetric label. In some embodiments, the reference detectable label is a radioactive label. In some embodiments, the reference detectable label is a luminescent label. In some embodiments, the reference detectable label is a chemiluminescent label. In some embodiments, the reference detectable label is a magnetic particle. In some embodiments, the reference detectable label is a metal particle. In some embodiments, the reference detectable label is a charged particle. In some embodiments, the reference detectable label is an ionic solution. In some embodiments, the reference detectable label is a spore. In some embodiments, the reference detectable label is an enzymatic label.

In some embodiments, the test subject is a mammal. In some embodiments, the test subject is a human or a non-human primate. In some embodiments, the test subject is human. In some embodiments, the test subject is a non-human primate. In some embodiments, the test subject is a monkey. In some embodiments, the test subject is a lab animal. In some embodiments, the test subject is a pig. In some embodiments, the test subject is a rat. In some embodiments, the test subject is a mouse. In some embodiments, the test subject is a cat. In some embodiments, the test subject is a dog. In some embodiments, the test subject is a guinea pig. In some embodiments, an AAV (e.g. AAV6) vector was administered to the test subject before the sample was acquired from the subject.

In some embodiments, the reference subject is a mammal. In some embodiments, the reference subject is a human or a non-human primate. In some embodiments, the reference subject is human. In some embodiments, the reference subject is a non-human primate. In some embodiments, the reference subject is a monkey. In some embodiments, the reference subject is a lab animal. In some embodiments, the reference subject is a pig. In some embodiments, the reference subject is a rat. In some embodiments, the reference subject is a mouse. In some embodiments, the reference subject is a cat. In some embodiments, the reference subject is a dog. In some embodiments, the reference subject is a guinea pig. In some embodiments, the test subject and the reference subject are the same species.

The skilled artisan would recognize that the test secondary antibody should specifically bind to the antibodies produced by the test subject. In some embodiments, the test secondary antibody is an anti-human antibody. Similarly, in some embodiments, the test secondary antibody may be anti-monkey antibody. In some embodiments, the test secondary antibody is an anti-pig antibody. In some embodiments, the test secondary antibody is an anti-rat antibody. In some embodiments, the test secondary antibody is an anti-mouse antibody. In some embodiments, the test secondary antibody is an anti-cat antibody. In some embodiments, the test secondary antibody is an anti-dog antibody. In some embodiments, the test secondary antibody is an anti-guinea pig antibody. In some embodiments, the test secondary antibody is an IgM, IgG, IgA, IgD, or IgG antibody. In some embodiments, the test secondary antibody is an anti-IgG antibody. In some embodiments, the test secondary antibody is an anti-IgM antibody. In some embodiments, the test secondary antibody is an anti-IgE antibody. In some embodiments, the test secondary antibody is an anti-IgA antibody. In some embodiments, the test secondary antibody is an anti-IgD antibody. In some embodiments, the test secondary antibody is an anti-human IgG.

The skilled artisan would recognize that the reference secondary antibody should specifically bind to the antibodies produced by the reference subject. In some embodiments, the reference secondary antibody is an anti-human antibody. Similarly, in some embodiments, the reference secondary antibody may be anti-monkey antibody. In some embodiments, the reference secondary antibody is an anti-pig antibody. In some embodiments, the reference secondary antibody is an anti-rat antibody. In some embodiments, the reference secondary antibody is an anti-mouse antibody. In some embodiments, the reference secondary antibody is an anti-cat antibody. In some embodiments, the reference secondary antibody is an anti-dog antibody. In some embodiments, the reference secondary antibody is an anti-guinea pig antibody. In some embodiments, the reference secondary antibody is an IgM, IgG, IgA, IgD, or IgG antibody. In some embodiments, the reference secondary antibody is an anti-IgG antibody. In some embodiments, the reference secondary antibody is an anti-IgM antibody. In some embodiments, the reference secondary antibody is an anti-IgE antibody. In some embodiments, the reference secondary antibody is an anti-IgA antibody. In some embodiments, the reference secondary antibody is an anti-IgD antibody. In some embodiments, the reference secondary antibody is an anti-human IgG. In some embodiments, the test secondary antibody and the reference secondary antibody are the same.

In some embodiments, the test solid support is a well of a culture plate. In some embodiments, the reference solid support is a well of a culture plate.

In some embodiments, the test immobilized AAV (e.g. AAV6) capsid comprises a polynucleotide. In some embodiments, the test immobilized AAV (e.g. AAV6) capsid does not comprise a polynucleotide. In some embodiments, the reference immobilized AAV (e.g. AAV6) capsid comprises a polynucleotide. In some embodiments, the reference immobilized AAV (e.g. AAV6) capsid does not comprise a polynucleotide.

Any anti-AAV antibody may be detected by any of the methods disclosed herein. In some embodiments, the anti-AAV antibody is selected from the group consisting of an anti-AAV1 antibody, an anti-AAV2 antibody, an anti-AAV3B antibody, an anti-AAV3 antibody, an anti-AAV4 antibody, an anti-AAV5 antibody, an anti-AAV6 antibody, an anti-AAV7 antibody, an anti-AAV8 antibody, an anti-AAV9 antibody, an anti-AAV10 antibody, an anti-AAV11 antibody, an anti-AAVrh10 antibody, an anti-AAVrh39 antibody, and an anti-AAVrh74 antibody. In some embodiments, the anti-AAV antibody is an anti-AAV6 antibody. In some embodiments, the test AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the test AAV capsid is an AAV6 capsid. The skilled artisan would recognize that the test AAV capsid should correspond to the anti-AAV antibody being detected. In some embodiments, the reference AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the reference AAV capsid is an AAV6 capsid. In some embodiments, the test AAV capsid is the same as the reference AAV capsid.

In some embodiments, the method comprises (a) providing a sample obtained from the subject; (b) contacting the sample with an AAV (e.g. AAV6) capsid, wherein the AAV (e.g. AAV6) capsid has been immobilized on a solid support; (c) contacting the sample-contacted immobilized AAV (e.g. AAV6) capsid with a secondary antibody, wherein the secondary antibody comprises a detectable label; and (d) detecting the label; wherein if the label is detected with a normalized response relative to a negative control of about 1.5 or greater, then the subject comprises anti-AAV (e.g. anti-AAV6) antibodies; and wherein if the label is detected with a normalized response relative to a negative control less than about 1.5, then the subject does not comprise anti-AAV (e.g. anti-AAV6) antibodies.

In some embodiments, if the normalized response relative to the negative control is about 1.6 or greater, then the subject comprises anti-AAV (e.g. anti-AAV6) antibodies; and wherein if the normalized response relative to the negative control is less than about 1.6, then the subject does not comprise anti-AAV (e.g. anti-AAV6) antibodies. In some embodiments, if the normalized response relative to the negative control is about 1.7 or greater, then the subject comprises anti-AAV (e.g. anti-AAV6) antibodies; and wherein if the normalized response relative to the negative control is less than about 1.7, then the subject does not comprise anti-AAV (e.g. anti-AAV6) antibodies. In some embodiments, if the normalized response relative to the negative control is about 1.73 or greater, then the subject comprises anti-AAV (e.g. anti-AAV6) antibodies; and wherein if the normalized response relative to the negative control is less than about 1.73, then the subject does not comprise anti-AAV (e.g. anti-AAV6) antibodies. In some embodiments, if the normalized response relative to the negative control is about 1.75 or greater, then the subject comprises anti-AAV (e.g. anti-AAV6) antibodies; and wherein if the normalized response relative to the negative control is less than about 1.75, then the subject does not comprise anti-AAV (e.g. anti-AAV6) antibodies.

In some embodiments, if the normalized response relative to the negative control is 1.5 or greater, then the subject comprises anti-AAV (e.g. anti-AAV6) antibodies; and wherein if the normalized response relative to the negative control is less than 1.5, then the subject does not comprise anti-AAV (e.g. anti-AAV6) antibodies. In some embodiments, if the normalized response relative to the negative control is 1.6 or greater, then the subject comprises anti-AAV (e.g. anti-AAV6) antibodies; and wherein if the normalized response relative to the negative control is less than 1.6, then the subject does not comprise anti-AAV (e.g. anti-AAV6) antibodies. In some embodiments, if the normalized response relative to the negative control is 1.7 or greater, then the subject comprises anti-AAV (e.g. anti-AAV6) antibodies; and wherein if the normalized response relative to the negative control is less than 1.7, then the subject does not comprise anti-AAV (e.g. anti-AAV6) antibodies. In some embodiments, if the normalized response relative to the negative control is 1.73 or greater, then the subject comprises anti-AAV (e.g. anti-AAV6) antibodies; and wherein if the normalized response relative to the negative control is less than 1.73, then the subject does not comprise anti-AAV (e.g. anti-AAV6) antibodies. In some embodiments, if the normalized response relative to the negative control is 1.75 or greater, then the subject comprises anti-AAV (e.g. anti-AAV6) antibodies; and wherein if the normalized response relative to the negative control is less than 1.75, then the subject does not comprise anti-AAV (e.g. anti-AAV6) antibodies.

In some embodiments, the sample comprises a fluid selected from the group consisting of serum, plasma, saliva, cerebral spinal fluid, and ocular fluid. In some embodiments, the sample comprises serum. In some embodiments, the sample comprises plasma. In some embodiments, the sample comprises saliva. In some embodiments, the sample comprises cerebral spinal fluid. In some embodiments, the sample comprises ocular fluid.

In some embodiments, the method further comprises the step of contacting the immobilized AAV (e.g. AAV6) capsid with a wash buffer after step (b) and before step (c). In some embodiments, the method further comprises the step of contacting the sample-contacted immobilized AAV (e.g. AAV6) capsid with a wash buffer after step (c) and before step (d). The wash buffer may comprise 1× phosphate buffered saline, 0.05% Tween 20. Tween 20 is also known as polysorbate 20. In some embodiments, the wash is repeated 3 times. In some embodiments, the wash is done using a plate washer. In some embodiments, the wash is done at room temperature.

In some embodiments, the detectable label is selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the detectable label is a horseradish peroxidase. In some embodiments, the detectable label is a fluorescent label. In some embodiments, the detectable label is a fluorogenic label. In some embodiments, the detectable label is a dye. In some embodiments, the detectable label is a colorimetric label. In some embodiments, the detectable label is a radioactive label. In some embodiments, the detectable label is a luminescent label. In some embodiments, the detectable label is a chemiluminescent label. In some embodiments, the detectable label is a magnetic particle. In some embodiments, the detectable label is a metal particle. In some embodiments, the detectable label is a charged particle. In some embodiments, the detectable label is an ionic solution. In some embodiments, the detectable label is a spore. In some embodiments, the detectable label is an enzymatic label. In some embodiments, the detectable label comprises of a combination of any of the detectable labels listed herein.

In some embodiments, the solid support is a well of a culture plate.

In some embodiments, the subject is a mammal. In some embodiments, the subject is a human or a non-human primate. In some embodiments, the subject is human. In some embodiments, the subject is a non-human primate. In some embodiments, the subject is a monkey. In some embodiments, the subject is a lab animal. In some embodiments, the subject is a pig. In some embodiments, the subject is a rat. In some embodiments, the subject is a mouse. In some embodiments, the subject is a cat. In some embodiments, the subject is a dog. In some embodiments, the subject is a guinea pig. In some embodiments, an AAV (e.g. AAV6) vector was administered to the subject before the sample was acquired from the subject.

The skilled artisan would recognize that the secondary antibody should specifically bind to the antibodies produced by the subject. In some embodiments, the secondary antibody is an anti-human antibody. Similarly, in some embodiments the secondary antibody may be anti-monkey antibody. In some embodiments, the secondary antibody is an anti-pig antibody. In some embodiments, the secondary antibody is an anti-rat antibody. In some embodiments, the secondary antibody is an anti-mouse antibody. In some embodiments, the secondary antibody is an anti-cat antibody. In some embodiments, the secondary antibody is an anti-dog antibody. In some embodiments, the secondary antibody is an anti-guinea pig antibody. In some embodiments, the secondary antibody is an IgM, IgG, IgA, IgD, or IgG antibody. In some embodiments, the secondary antibody is an anti-IgG antibody. In some embodiments, the secondary antibody is an anti-IgM antibody. In some embodiments, the secondary antibody is an anti-IgE antibody. In some embodiments, the secondary antibody is an anti-IgA antibody. In some embodiments, the secondary antibody is an anti-IgD antibody. In some embodiments, the secondary antibody is an anti-human IgG.

In some embodiments, the immobilized AAV (e.g. AAV6) capsid comprises a polynucleotide. In some embodiments, the immobilized AAV (e.g. AAV6) capsid does not comprise a polynucleotide.

Any anti-AAV antibody may be detected by the methods disclosed herein. In some embodiments, the anti-AAV antibody is selected from the group consisting of an anti-AAV1 antibody, an anti-AAV2 antibody, an anti-AAV3B antibody, an anti-AAV3 antibody, an anti-AAV4 antibody, an anti-AAV5 antibody, an anti-AAV6 antibody, an anti-AAV7 antibody, an anti-AAV8 antibody, an anti-AAV9 antibody, an anti-AAV10 antibody, an anti-AAV11 antibody, an anti-AAVrh10 antibody, an anti-AAVrh39 antibody, and an anti-AAVrh74 antibody. In some embodiments, the anti-AAV antibody is an anti-AAV6 antibody. In some embodiments, the AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the AAV capsid is an AAV6 capsid. The skilled artisan would recognize that the AAV capsid should correspond to the anti-AAV antibody being detected.

In some embodiments of any of the methods disclosed herein, the negative control is a pre-screened negative sample. In some embodiments of any of the methods disclosed herein, the negative control was negative in a neutralizing antibody assay. See, e.g., FIG. 1A.

In some embodiments of any of the methods disclosed herein, the screening assay is an ELISA. In some embodiments, the ELISA comprises the following steps:
1. Coating ELISA plates with 100 μL/well of diluted AAV (e.g. AAV6) capsids (1.5E10 cp/mL),4° C. overnight
2. Washing plates 3 times with 300 μL phosphate buffer saline with Tween 20.
3. Adding 200 μL of sample buffer to all wells and incubating for 2-3 hours at room temperature.
4. Washing plates 3 times with 300 μL phosphate buffer saline with Tween 20.
5. Adding 100 μL samples and incubating for 2 hours at room temperature.
6. Washing plates 6 times with 300 μL phosphate buffer saline with Tween 20.
7. Adding 100 μL of goat anti-human IgG conjugated to horseradish peroxidase (Jackson, 0.8 mg/ml 1:40k dilution) and incubating for 1 hour at room temperature.
8. Washing plates 3 times with 300 μL phosphate buffer saline with Tween 20.
9. Adding 100 μL of 3, 3′, 5, 5′-tetramethylbenzidine (TMB) and incubating for 15 minutes at room temperature.
10. Adding 100 μL of Stop Solution and reading plates at 450/650 nm.
   In some embodiments, the sample buffer is 0.5% bovine serum albumin in phosphate buffered saline with 0.05% Tween 20.

### 4. Detection of Anti-AAV (e.g. anti-AAV6) Antibodies Using A Confirmatory assay

A second aspect of the present disclosure provides a method of detecting anti-AAV (e.g. anti-AAV6) antibodies in a subject using a confirmatory assay.

In some embodiments, the method comprises (a) providing a first test sample and a second test sample obtained from the test subject; (b) contacting the second test sample with a test soluble AAV capsid; (c) contacting the first test sample with a first test immobilized AAV capsid, wherein the first test immobilized AAV capsid has been immobilized on a first test solid support; (d) contacting the mixture of the second test sample and the test soluble AAV capsid with a second test immobilized AAV capsid, wherein the second test immobilized AAV capsid has been immobilized on a second test solid support; (e) contacting the first test sample-contacted first test immobilized AAV capsid with a first test secondary antibody, wherein the first test secondary antibody comprises a first test detectable label; (f) contacting the mixture-contacted second test immobilized AAV capsid with a second test secondary antibody, wherein the second test secondary antibody comprises a second test detectable label, wherein the second test secondary antibody is the same as the first test secondary antibody and the first test detectable label is the same as the second test detectable label; and (g) detecting the first and second test detectable labels; wherein if the amount of second label detected is reduced by a cut point percentage or more compared to the amount of first label detected, then the subject comprises anti-AAV antibodies; and wherein if the amount of second label detected is reduced by less than the cut point compared to the amount of first label detected, then the subject does not comprise anti-AAV antibodies.

In some embodiments, the cut point percentage is determined by a method comprising: (1) providing first and second reference samples obtained from a plurality of reference subjects, wherein each of the reference subjects was negative for a neutralizing anti-AAV antibody assay; (2) contacting, for each reference subject, the second reference sample with a reference soluble AAV capsid; (3) contacting, for each reference subject, the first reference sample with a first reference immobilized AAV capsid, wherein the first reference immobilized AAV capsid has been immobilized on a first reference solid support; (4) contacting, for each reference subject, the mixture of the second reference sample and the reference soluble AAV capsid with a second reference immobilized AAV capsid, wherein the second reference immobilized AAV capsid has been immobilized on a second reference solid support; (5) contacting, for each reference subject, the first reference sample-contacted first immobilized AAV capsid with a first secondary antibody, wherein the first secondary antibody comprises a first reference detectable label; (6) contacting, for each reference subject, the mixture-contacted second immobilized AAV capsid with a second secondary antibody, wherein the second secondary antibody comprises a second reference detectable label, wherein the second secondary antibody is the same as the first secondary antibody and the first reference detectable label is the same as the second reference detectable label; (7) detecting, for each reference subject, the first and second reference detectable labels; (8) removing outlier reference samples; and (9) using the outlier-removed reference samples to determine the cut point percentage.

In some embodiments, the outlier reference samples are removed using the criteria [Q1−1.5*(Q3−Q1),Q3+1.5*(Q3−Q1)]. In some embodiments, the use of prescreened negative subjects for determining the cut point percentage results in at least one fewer round of outlier removal compared to the use of random subjects for determining the cut point percentage. In some embodiments, the cut point percentage is based on a parametric method with a 95% confidence interval (i.e. is equal to the mean+(1.645×SD)). In some embodiments, the cut point percentage is based on a parametric method with a 99% confidence interval (i.e. is equal to the mean+(2.33×SD)). In some embodiments, the cut point percentage is based on a robust parametric method with a 95% confidence interval (i.e. is equal to the median+(1.645×(1.483×MAD))). In some embodiments, the cut point percentage is based on a robust parametric method with a 99% confidence interval (i.e. is equal to the median+(2.33×SD)). In some embodiments, the cut point percentage is based on a non-parametric method (i.e. the 99th percentile).

In some embodiments, the anti-AAV antibodies are anti-AAV6 antibodies, the test soluble AAV capsid is a soluble test AAV6 capsid, the first test immobilized AAV capsid is a first test immobilized AAV6 capsid, and the second test immobilized AAV capsid is a second test immobilized AAV6 capsid. In some embodiments, the anti-AAV antibodies are anti-AAV6 antibodies, the reference soluble AAV capsid is a soluble reference AAV6 capsid, the first reference immobilized AAV capsid is a first reference immobilized AAV6 capsid, and the second reference immobilized AAV capsid is a second reference immobilized AAV6 capsid.

In some embodiments, the first and second test sample each comprise a fluid selected from the group consisting of serum, plasma, saliva, cerebral spinal fluid, and ocular fluid. In some embodiments, the first and second test sample each comprise serum. In some embodiments, the first and second test sample each comprise plasma. In some embodiments, the first and second test sample each comprise saliva. In some embodiments, the first and second test sample each comprise cerebral spinal fluid. In some embodiments, the first and second test sample each comprise ocular fluid.

In some embodiments, each first and second reference sample comprises a fluid selected from the group consisting of serum, plasma, saliva, cerebral spinal fluid, and ocular fluid. In some embodiments, the first and second reference sample each comprise serum. In some embodiments, the first and second reference sample each comprise plasma. In some embodiments, the first and second reference sample each comprise saliva. In some embodiments, the first and second reference sample each comprise cerebral spinal fluid. In some embodiments, the first and second reference sample each comprise ocular fluid.

In some embodiments, the method further comprises the step of contacting the first test immobilized AAV6 capsid with a wash buffer after step (c) and before step (e). In some embodiments, the method further comprises the step of contacting the second test immobilized AAV6 capsid with a wash buffer after step (d) and before step (f). In some embodiments, the method further comprises the step of contacting the sample-contacted first test immobilized AAV6 capsid with a wash buffer after step (e) and before step (g). In some embodiments, the method further comprises the step of contacting the mixture-contacted second test immobilized AAV6 capsid with a wash buffer after step (f) and before step (g). The wash buffer may comprise 1× phosphate buffered saline, 0.05% Tween 20. Tween 20 is also known as polysorbate 20. In some embodiments, the wash is repeated 3 times. In some embodiments, the wash is done using a plate washer. In some embodiments, the wash is done at room temperature.

In some embodiments, the method further comprises the step of contacting the first reference immobilized AAV6 capsid with a wash buffer after step (3) and before step (5). In some embodiments, the method further comprises the step of contacting the second reference immobilized AAV6 capsid with a wash buffer after step (4) and before step (6). In some embodiments, the method further comprises the step of contacting the sample-contacted first reference immobilized AAV6 capsid with a wash buffer after step (5) and before step (7). In some embodiments, the method further comprises the step of contacting the mixture-contacted second reference immobilized AAV6 capsid with a wash buffer after step (6) and before step (8). The wash buffer may comprise 1× phosphate buffered saline, 0.05% Tween 20. Tween 20 is also known as polysorbate 20. In some embodiments, the wash is repeated 3 times. In some embodiments, the wash is done using a plate washer. In some embodiments, the wash is done at room temperature.

In some embodiments, the first and second test detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the first and second test detectable label are fluorescent labels. In some embodiments, the first and second test detectable label are fluorogenic labels. In some embodiments, the first and second test detectable label are dyes. In some embodiments, the first and second test detectable label are colorimetric labels. In some embodiments, the first and second test detectable label are radioactive labels. In some embodiments, the first and second test detectable label are luminescent labels. In some embodiments, the first and second test detectable label are chemiluminescent labels. In some embodiments, the first and second test detectable label are magnetic particles. In some embodiments, the first and second test detectable label are metal particles. In some embodiments, the first and second test detectable label are charged particles. In some embodiments, the first and second test detectable label are ionic solutions. In some embodiments, the first and second test detectable label are spores. In some embodiments, the first and second test detectable label are enzymatic labels.

In some embodiments, the first and second reference detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof. In some embodiments, the first and second reference detectable label are fluorescent labels. In some embodiments, the first and second reference detectable label are fluorogenic labels. In some embodiments, the first and second reference detectable label are dyes. In some embodiments, the first and second reference detectable label are colorimetric labels. In some embodiments, the first and second reference detectable label are radioactive labels. In some embodiments, the first and second reference detectable label are luminescent labels. In some embodiments, the first and second reference detectable label are chemiluminescent labels. In some embodiments, the first and second reference detectable label are magnetic particles. In some embodiments, the first and second reference detectable label are metal particles. In some embodiments, the first and second reference detectable label are charged particles. In some embodiments, the first and second reference detectable label are ionic solutions. In some embodiments, the first and second reference detectable label are spores. In some embodiments, the first and second reference detectable label are enzymatic labels.

In some embodiments, the test subject is a mammal. In some embodiments, the test subject is a human or a non-human primate. In some embodiments, the test subject is human. In some embodiments, the test subject is a non-human primate. In some embodiments, the test subject is a monkey. In some embodiments, the test subject is a lab animal. In some embodiments, the test subject is a pig. In some embodiments, the test subject is a rat. In some embodiments, the test subject is a mouse. In some embodiments, the test subject is a cat. In some embodiments, the test subject is a dog. In some embodiments, the test subject is a guinea pig. In some embodiments, an AAV (e.g. AAV6) vector was administered to the test subject before the sample was acquired from the subject.

In some embodiments, the reference subject is a mammal. In some embodiments, the reference subject is a human or a non-human primate. In some embodiments, the reference subject is human. In some embodiments, the reference subject is a non-human primate. In some embodiments, the reference subject is a monkey. In some embodiments, the reference subject is a lab animal. In some embodiments, the reference subject is a pig. In some embodiments, the reference subject is a rat. In some embodiments, the reference subject is a mouse. In some embodiments, the reference subject is a cat. In some embodiments, the reference subject is a dog. In some embodiments, the reference subject is a guinea pig. In some embodiments, the test subject and the reference subject are the same species.

The skilled artisan would recognize that the first and second test secondary antibodies should specifically bind to the antibodies produced by the test subject. In some embodiments, the first and second test secondary antibodies are anti-human antibodies. Similarly, in some embodiments, the first and second test secondary antibodies are anti-monkey antibodies. In some embodiments, the first and second test secondary antibodies are anti-pig antibodies. In some embodiments, the first and second test secondary antibodies are anti-rat antibodies. In some embodiments, the first and second test secondary antibodies are anti-mouse antibodies. In some embodiments, the first and second test secondary antibodies are anti-cat antibodies. In some embodiments, the first and second test secondary antibodies are anti-dog antibodies. In some embodiments, the first and second test secondary antibodies are anti-guinea pig antibodies. In some embodiments, the first and second test secondary antibodies are IgM, IgG, IgA, IgD, or IgG antibodies. In some embodiments, the first and second test secondary antibodies are anti-IgG antibodies. In some embodiments, the first and second test secondary antibodies are anti-IgM antibodies. In some embodiments, the first and second test secondary antibodies are anti-IgE antibodies. In some embodiments, the first and second test secondary antibodies are anti-IgA antibodies. In some embodiments, the first and second test secondary antibodies are anti-IgD antibodies. In some embodiments, the first and second test secondary antibodies are anti-human IgG antibodies. In some embodiments the first and second test secondary antibodies are the same.

The skilled artisan would recognize that the first and second reference secondary antibodies should specifically bind to the antibodies produced by the reference subject. In some embodiments, the first and second reference secondary antibodies are anti-human antibodies. Similarly, in some embodiments, the first and second reference secondary antibodies are anti-monkey antibodies. In some embodiments, the first and second reference secondary antibodies are anti-pig antibodies. In some embodiments, the first and second reference secondary antibodies are anti-rat antibodies. In some embodiments, the first and second reference secondary antibodies are anti-mouse antibodies. In some embodiments, the first and second reference secondary antibodies are anti-cat antibodies. In some embodiments, the first and second reference secondary antibodies are anti-dog antibodies. In some embodiments, the first and second reference secondary antibodies are anti-guinea pig antibodies. In some embodiments, the first and second reference secondary antibodies are IgM, IgG, IgA, IgD, or IgG antibodies. In some embodiments, the first and second reference secondary antibodies are anti-IgG antibodies. In some embodiments, the first and second reference secondary antibodies are anti-IgM antibodies. In some embodiments, the first and second reference secondary antibodies are anti-IgE antibodies. In some embodiments, the first and second reference secondary antibodies are anti-IgA antibodies. In some embodiments, the first and second reference secondary antibodies are anti-IgD antibodies. In some embodiments, the first and second reference secondary antibodies are anti-human IgG antibodies. In some embodiments the first and second reference secondary antibodies are the same. In some embodiments, the first and second test secondary antibodies are the same as the first and second the reference secondary antibodies.

In some embodiments, the first test solid support is a well of a culture plate. In some embodiments, the second test solid support is a well of a culture plate. In some embodiments, the first reference solid support is a well of a culture plate. In some embodiments, the second reference solid support is a well of a culture plate.

In some embodiments, the first test immobilized AAV (e.g. AAV6) capsid comprises a polynucleotide. In some embodiments, the first test immobilized AAV (e.g. AAV6) capsid does not comprise a polynucleotide. In some embodiments, the first second immobilized AAV (e.g. AAV6) capsid comprises a polynucleotide. In some embodiments, the second test immobilized AAV (e.g. AAV6) capsid does not comprise a polynucleotide. In some embodiments, the first reference immobilized AAV (e.g. AAV6) capsid comprises a polynucleotide. In some embodiments, the first reference immobilized AAV (e.g. AAV6) capsid does not comprise a polynucleotide. In some embodiments, the second reference immobilized AAV (e.g. AAV6) capsid comprises a polynucleotide. In some embodiments, the second reference immobilized AAV (e.g. AAV6) capsid does not comprise a polynucleotide.

Any anti-AAV antibody may be detected by any of the methods disclosed herein. In some embodiments, the anti-AAV antibody is selected from the group consisting of an anti-AAV1 antibody, an anti-AAV2 antibody, an anti-AAV3B antibody, an anti-AAV3 antibody, an anti-AAV4 antibody, an anti-AAV5 antibody, an anti-AAV6 antibody, an anti-AAV7 antibody, an anti-AAV8 antibody, an anti-AAV9 antibody, an anti-AAV10 antibody, an anti-AAV11 antibody, an anti-AAVrh10 antibody, an anti-AAVrh39 antibody, and an anti-AAVrh74 antibody. In some embodiments, the anti-AAV antibody is an anti-AAV6 antibody. In some embodiments, the first test AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the first test AAV capsid is an AAV6 capsid. The skilled artisan would recognize that the first test AAV capsid should correspond to the anti-AAV antibody being detected. In some embodiments, the second test AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the second test AAV capsid is an AAV6 capsid. The skilled artisan would recognize that the second test AAV capsid should correspond to the anti-AAV antibody being detected. In some embodiments, the first test AAV capsid is the same as the second test AAV capsid.

In some embodiments, the first reference AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the first reference AAV capsid is an AAV6 capsid. In some embodiments, the second reference AAV capsid is selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some embodiments, the second reference AAV capsid is an AAV6 capsid. In some embodiments, the first reference AAV capsid is the same as the second reference AAV capsid. In some embodiments, the first and second test AAV capsid are the same as the first and second reference AAV capsid.

In some embodiments, the test soluble AAV (e.g. AAV6) capsid is at a concentration of about 7.5E10-1.5E11 cp/mL. In some embodiments, the test soluble AAV (e.g. AAV6) capsid is at a concentration of about 7.5E10 cp/mL. In some embodiments, the test soluble AAV (e.g. AAV6) capsid is at a concentration of about 8E10 cp/mL. In some embodiments, the test soluble AAV (e.g. AAV6) capsid is at a concentration of about 9E10 cp/mL. In some embodiments, the test soluble AAV (e.g. AAV6) capsid is at a concentration of about 1.0E11 cp/mL. In some embodiments, the test soluble AAV (e.g. AAV6) capsid is at a concentration of about 1.1E11 cp/mL. In some embodiments, the test soluble AAV (e.g. AAV6) capsid is at a concentration of about 1.2E11 cp/mL. In some embodiments, the test soluble AAV (e.g. AAV6) capsid is at a concentration of about 1.3E11 cp/mL. In some embodiments, the test soluble AAV (e.g. AAV6) capsid is at a concentration of about 1.4E11 cp/mL. In some embodiments, the test soluble AAV (e.g. AAV6) capsid is at a concentration of about 1.5E11 cp/mL.

In some embodiments, the test soluble AAV (e.g. AAV6) capsid is at a concentration of 7.5E10-1.5E11 cp/mL. In some embodiments, the test soluble AAV (e.g. AAV6) capsid is at a concentration of 7.5E10 cp/mL. In some embodiments, the test soluble AAV (e.g. AAV6) capsid is at a concentration of 8E10 cp/mL. In some embodiments, the test soluble AAV (e.g. AAV6) capsid is at a concentration of 9E10 cp/mL. In some embodiments, the test soluble AAV (e.g. AAV6) capsid is at a concentration of 1.0E11 cp/mL. In some embodiments, the test soluble AAV (e.g. AAV6) capsid is at a concentration of 1.1E11 cp/mL. In some embodiments, the test soluble AAV (e.g. AAV6) capsid is at a concentration of 1.2E11 cp/mL. In some embodiments, the test soluble AAV (e.g. AAV6) capsid is at a concentration of 1.3E11 cp/mL. In some embodiments, the test soluble AAV (e.g. AAV6) capsid is at a concentration of 1.4E11 cp/mL. In some embodiments, the test soluble AAV (e.g. AAV6) capsid is at a concentration of 1.5E11 cp/mL. In some embodiments, the reference soluble AAV (e.g. AAV6) capsid is at a concentration of about 7.5E10-1.5E11 cp/mL. In some embodiments, the reference soluble AAV (e.g. AAV6) capsid is at a concentration of about 7.5E10 cp/mL. In some embodiments, the reference soluble AAV (e.g. AAV6) capsid is at a concentration of about 8E10 cp/mL. In some embodiments, the reference soluble AAV (e.g. AAV6) capsid is at a concentration of about 9E10 cp/mL. In some embodiments, the reference soluble AAV (e.g. AAV6) capsid is at a concentration of about 1.0E11 cp/mL. In some embodiments, the reference soluble AAV (e.g. AAV6) capsid is at a concentration of about 1.1E11 cp/mL. In some embodiments, the reference soluble AAV (e.g. AAV6) capsid is at a concentration of about 1.2E11 cp/mL. In some embodiments, the reference soluble AAV (e.g. AAV6) capsid is at a concentration of about 1.3E11 cp/mL. In some embodiments, the reference soluble AAV (e.g. AAV6) capsid is at a concentration of about 1.4E11 cp/mL. In some embodiments, the reference soluble AAV (e.g. AAV6) capsid is at a concentration of about 1.5E11 cp/mL.

In some embodiments, the reference soluble AAV (e.g. AAV6) capsid is at a concentration of 7.5E10-1.5E11 cp/mL. In some embodiments, the reference soluble AAV (e.g. AAV6) capsid is at a concentration of 7.5E10 cp/mL. In some embodiments, the reference soluble AAV (e.g. AAV6) capsid is at a concentration of 8E10 cp/mL. In some embodiments, the reference soluble AAV (e.g. AAV6) capsid is at a concentration of 9E10 cp/mL. In some embodiments, the reference soluble AAV (e.g. AAV6) capsid is at a concentration of 1.0E11 cp/mL. In some embodiments, the reference soluble AAV (e.g. AAV6) capsid is at a concentration of 1.1E11 cp/mL. In some embodiments, the reference soluble AAV (e.g. AAV6) capsid is at a concentration of 1.2E11 cp/mL. In some embodiments, the reference soluble AAV (e.g. AAV6) capsid is at a concentration of 1.3E11 cp/mL. In some embodiments, the reference soluble AAV (e.g. AAV6) capsid is at a concentration of 1.4E11 cp/mL. In some embodiments, the reference soluble AAV (e.g. AAV6) capsid is at a concentration of 1.5E11 cp/mL.

In some embodiments, the method comprises (a) providing a first sample and a second sample obtained from the subject; (b) contacting the second sample with a soluble AAV (e.g. AAV6) capsid; (c) contacting the first sample with a first immobilized AAV (e.g. AAV6) capsid, wherein the first immobilized AAV (e.g. AAV6) capsid has been immobilized on a first solid support; (d) contacting the mixture of the second sample and the soluble AAV (e.g. AAV6) capsid with a second immobilized AAV (e.g. AAV6) capsid, wherein the second immobilized AAV (e.g. AAV6) capsid has been immobilized on a second solid support; (e) contacting the first sample-contacted first immobilized AAV (e.g. AAV6) capsid with a first secondary antibody, wherein the first secondary antibody comprises a first detectable label; (f) contacting the mixture-contacted second immobilized AAV (e.g. AAV6) capsid with a second secondary antibody, wherein the second secondary antibody comprises a second detectable label, wherein the second secondary antibody is the same as the first secondary antibody and the second detectable label is the same as the second detectable label; and (g) detecting the first and second labels; wherein if the amount of second label detected is reduced by 20% or more compared to the amount of first label detected, then the subject comprises anti-AAV (e.g. anti-AAV6) antibodies; and wherein if the amount of second label detected is reduced by less than 20% compared to the amount of first label detected, then the subject does not comprise anti-AAV (e.g. anti-AAV6) antibodies.

In some embodiments, if the amount of second label detected is reduced by about 22% or more compared to the amount of first label detected, then the subject comprises anti-AAV (e.g. anti-AAV6) antibodies; and wherein if the amount of second label detected is reduced by less than about 22% compared to the amount of first label detected, then the subject does not comprise anti-AAV (e.g. anti-AAV6) antibodies. In some embodiments, if the amount of second label detected is reduced by about 24% or more compared to the amount of first label detected, then the subject comprises anti-AAV (e.g. anti-AAV6) antibodies; and wherein if the amount of second label detected is reduced by less than about 24% compared to the amount of first label detected, then the subject does not comprise anti-AAV (e.g. anti-AAV6) antibodies. In some embodiments, if the amount of second label detected is reduced by about 24.1% or more compared to the amount of first label detected, then the subject comprises anti-AAV (e.g. anti-AAV6) antibodies; and wherein if the amount of second label detected is reduced by less than about 24.1% compared to the amount of first label detected, then the subject does not comprise anti-AAV (e.g. anti-AAV6) antibodies. In some embodiments, if the amount of second label detected is reduced by about 24.5% or more compared to the amount of first label detected, then the subject comprises anti-AAV (e.g. anti-AAV6) antibodies; and wherein if the amount of second label detected is reduced by less than about 24.5% compared to the amount of first label detected, then the subject does not comprise anti-AAV (e.g. anti-AAV6) antibodies.

In some embodiments, if the amount of second label detected is reduced by 22% or more compared to the amount of first label detected, then the subject comprises anti-AAV (e.g. anti-AAV6) antibodies; and wherein if the amount of second label detected is reduced by less than 22% compared to the amount of first label detected, then the subject does not comprise anti-AAV (e.g. anti-AAV6) antibodies. In some embodiments, if the amount of second label detected is reduced by 24% or more compared to the amount of first label detected, then the subject comprises anti-AAV (e.g. anti-AAV6) antibodies; and wherein if the amount of second label detected is reduced by less than 24% compared to the amount of first label detected, then the subject does not comprise anti-AAV (e.g. anti-AAV6) antibodies. In some embodiments, if the amount of second label detected is reduced by 24.1% or more compared to the amount of first label detected, then the subject comprises anti-AAV (e.g. anti-AAV6) antibodies; and wherein if the amount of second label detected is reduced by less than 24.1% compared to the amount of first label detected, then the subject does not comprise anti-AAV (e.g. anti-AAV6) antibodies. In some embodiments, if the amount of second label detected is reduced by 24.5% or more compared to the amount of first label detected, then the subject comprises anti-AAV (e.g. anti-AAV6) antibodies; and wherein if the amount of second label detected is reduced by less than 24.5% compared to the amount of first label detected, then the subject does not comprise anti-AAV (e.g. anti-AAV6) antibodies.

In some embodiments, the first and second sample each comprises a fluid selected from the group consisting of serum, plasma, saliva, cerebral spinal fluid, and ocular fluid. The first sample may be the same as the second sample. In some embodiments, the first and second sample each comprises serum. In some embodiments, the first and second sample each comprises plasma. In some embodiments, the first and second sample each comprises saliva.

In some embodiments, the first and second sample each comprises cerebral spinal fluid. In some embodiments, the first and second sample each comprises ocular fluids. In some embodiments, the first and second samples are derived from the common sample.

In some embodiments, the method further comprises the step of contacting the first immobilized AAV (e.g. AAV6) capsid with a wash buffer after step (c) and before step (e). In some embodiments, the method further comprises the step of contacting the second immobilized AAV (e.g. AAV6) capsid with a wash buffer after step (d) and before step (f). In some embodiments, the method further comprises the step of contacting the sample-contacted first immobilized AAV (e.g. AAV6) capsid with a wash buffer after step (e) and before step (g). In some embodiments, the method further comprises the step of contacting the mixture-contacted second immobilized AAV (e.g. AAV6) capsid with a wash buffer after step (f) and before step (g). The wash buffer may comprise 1× phosphate buffered saline, 0.05% Tween 20. In some embodiments, the wash is repeated 3 times. In some embodiments, the wash is done using a plate washer. In some embodiments, the wash is done at room temperature.

In some embodiments, the first and second detectable label are each selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof In some embodiments, the first detectable label is the same as the second detectable label. In some embodiments, the first and second detectable label are each horseradish peroxidase. In some embodiments, the first and second detectable label are each a fluorescent label. In some embodiments, the first and second detectable label are each a fluorogenic label. In some embodiments, the first and second detectable label are each a dye. In some embodiments, the first and second detectable label are each a colorimetric label. In some embodiments, the first and second detectable label are each a radioactive label. In some embodiments, the first and second detectable label are each a luminescent label. In some embodiments, the first and second detectable label are each a chemiluminescent label. In some embodiments, the first and second detectable label are each a magnetic particle. In some embodiments, the first and second detectable label are each a metal particle. In some embodiments, the first and second detectable label are each a charged particle. In some embodiments, the first and second detectable label are each an ionic solution. In some embodiments, the first and second detectable label are each a spore. In some embodiments, the first and second detectable label are each an enzymatic label. In some embodiments, the first and second detectable label each comprises a combination of any of the detectable labels listed herein.

In some embodiments, the first and second solid support are each a well of a culture plate.

In some embodiments, the subject is a mammal. In some embodiments, the subject is a human or a non-human primate. In some embodiments, the subject is a human. In some embodiments, the subject is a non-human primate. In some embodiments, the subject is a monkey. In some embodiments, the subject is a lab animal. In some embodiments, the subject is a pig. In some embodiments, the subject is a rat. In some embodiments, the subject is a mouse. In some embodiments, the subject is a cat. In some embodiments, the subject is a dog. In some embodiments, the subject is a guinea pig. In some embodiments, an AAV (e.g. AAV6) vector was administered to the subject before the first and second sample were acquired from the subject.

The skilled artisan would recognize that the first and second secondary antibodies should specifically bind to the antibodies produced by the subject. In some embodiments, the first and second secondary antibodies are each IgM, IgG, IgA, IgD, or IgG antibodies. In some embodiments, the first and second secondary antibodies are each anti-IgG antibodies. In some embodiments, the first and second secondary antibodies are each anti-IgM antibodies. In some embodiments, the first and second secondary antibodies are each anti-IgE antibodies. In some embodiments, the first and second secondary antibodies are each anti-IgA antibodies. In some embodiments, the first and second secondary antibodies are each anti-IgD antibodies. In some embodiments, the first and second secondary antibodies are each an anti-human IgG antibody. Similarly, in some embodiments, the first and second secondary antibodies are each an anti-monkey antibody. In some embodiments, the first and second secondary antibodies are each an anti-pig antibody. In some embodiments, the first and second secondary antibody are each an anti-rat antibody. In some embodiments, the first and second secondary antibodies are each an anti-mouse antibody. In some embodiments, the first and second secondary antibodies are each an anti-cat antibody. In some embodiments, the first and second secondary antibodies are each an anti-dog antibody. In some embodiments, the first and second secondary antibodies are each an anti-guinea pig antibody.

In some embodiments, the soluble AAV (e.g. AAV6) capsid is at a concentration of about 7.5E10-1.5E11 cp/mL. In some embodiments, the soluble AAV (e.g. AAV6) capsid is at a concentration of about 7.5E10 cp/mL. In some embodiments, the soluble AAV (e.g. AAV6) capsid is at a concentration of about 8E10 cp/mL. In some embodiments, the soluble AAV (e.g. AAV6) capsid is at a concentration of about 9E10 cp/mL. In some embodiments, the soluble AAV (e.g. AAV6) capsid is at a concentration of about 1.0E11 cp/mL. In some embodiments, the soluble AAV (e.g. AAV6) capsid is at a concentration of about 1.1E11 cp/mL. In some embodiments, the soluble AAV (e.g. AAV6) capsid is at a concentration of about 1.2E11 cp/mL. In some embodiments, the soluble AAV (e.g. AAV6) capsid is at a concentration of about 1.3E11 cp/mL. In some embodiments, the soluble AAV (e.g. AAV6) capsid is at a concentration of about 1.4E11 cp/mL. In some embodiments, the soluble AAV (e.g. AAV6) capsid is at a concentration of about 1.5E11 cp/mL.

In some embodiments, the soluble AAV (e.g. AAV6) capsid is at a concentration of 7.5E10-1.5E11 cp/mL. In some embodiments, the soluble AAV (e.g. AAV6) capsid is at a concentration of 7.5E10 cp/mL. In some embodiments, the soluble AAV (e.g. AAV6) capsid is at a concentration of 8E10 cp/mL. In some embodiments, the soluble AAV (e.g. AAV6) capsid is at a concentration of 9E10 cp/mL. In some embodiments, the soluble AAV (e.g. AAV6) capsid is at a concentration of 1.0E11 cp/mL. In some embodiments, the soluble AAV (e.g. AAV6) capsid is at a concentration of 1.1E11 cp/mL. In some embodiments, the soluble AAV (e.g. AAV6) capsid is at a concentration of 1.2E11 cp/mL. In some embodiments, the soluble AAV (e.g. AAV6) capsid is at a concentration of 1.3E11 cp/mL. In some embodiments, the soluble AAV (e.g. AAV6) capsid is at a concentration of 1.4E11 cp/mL. In some embodiments, the soluble AAV (e.g. AAV6) capsid is at a concentration of 1.5E11 cp/mL.

In some embodiments, the first immobilized AAV (e.g. AAV6) capsid comprises a polynucleotide. In some embodiments, the first immobilized AAV (e.g. AAV6) capsid does not comprise a polynucleotide. In some embodiments, the second immobilized AAV (e.g. AAV6) capsid comprises a polynucleotide. In some embodiments, the second immobilized AAV (e.g. AAV6) capsid does not comprise a polynucleotide. In some embodiments, the soluble AAV (e.g. AAV6) capsid comprises a polynucleotide. In some embodiments, the soluble AAV (e.g. AAV6) capsid does not comprise a polynucleotide.

In some embodiments, the confirmatory assay is an ELISA. In some embodiments, the challenge ELISA comprises the following steps:
1. Coating ELISA plates with 100 μL/well of diluted AAV (e.g. AAV6) capsids (1.5e10 cp/mL),4° C. overnight
2. Washing plates 3 times with 300 μL phosphate buffer saline with Tween 20.
3. Adding 200 μL of sample buffer to all wells and incubating for 2-3 hours at room temperature.
4. Washing plates 3 times with 300 μL phosphate buffer saline with Tween 20.
5. Adding 7.5e10 cp/ml of soluble AAV (e.g. AAV6) capsid to one of two samples obtained from a subject.
6. Adding 100 μL samples and incubating for 2 hours at room temperature.
7. Washing plates 6 times with 300 μL phosphate buffer saline with Tween 20.
8. Adding 100 μL of goat anti-human IgG conjugated to horseradish peroxidase (Jackson, 0.8 mg/ml 1:40k dilution) and incubating for 1 hour at room temperature.
9. Washing plates 3 times with 300 μL phosphate buffer saline with Tween 20.
10. Adding 100 μL of 3, 3′, 5, 5′-tetramethylbenzidine (TMB) and incubating for 15 minutes at room temperature.
11. Adding 100 μL of Stop Solution and reading plates at 450/650 nm.

In some embodiments, the sample buffer is 0.5% bovine serum albumin in phosphate buffered saline with 0.05% Tween 20.

Any anti-AAV antibody may be detected by the methods disclosed herein. In some embodiments, the anti-AAV antibody is selected from the group consisting of an anti-AAV1 antibody, an anti-AAV2 antibody, an anti-AAV3B antibody, an anti-AAV3 antibody, an anti-AAV4 antibody, an anti-AAV5 antibody, an anti-AAV6 antibody, an anti-AAV7 antibody, an anti-AAV8 antibody, an anti-AAV9 antibody, an anti-AAV10 antibody, an anti-AAV11 antibody, an anti-AAVrh10 antibody, an anti-AAVrh39 antibody, and an anti-AAVrh74 antibody. In some embodiments, the anti-AAV antibody is an anti-AAV6 antibody. In some embodiments, the soluble AAV capsid, the first immobilized AAV capsid, and the second immobilized AAV capsid are each selected from the group consisting of an AAV1 capsid, an AAV2 capsid, an AAV3B capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAVrh10 capsid, an AAVrh39 capsid, and an AAVrh74 capsid. In some 15 embodiments, the soluble AAV capsid, the first immobilized AAV capsid, and the second immobilized AAV capsid are each an AAV6 capsid. The skilled artisan would recognize that the soluble AAV capsid, the first immobilized AAV capsid and the second immobilized AAV capsid should be the same and should correspond to the anti-AAV antibody being detected.

### 5. Methods of Treatment

A third aspect of the present disclosure provides a method of treating a subject in need of gene therapy. In some embodiments, the method comprises providing the subject and administering a recombinant adeno-associated virus (rAAV, for example a rAAV6) vector to the subject only if the subject does not comprise anti-AAV (e.g. anti-AAV6) antibodies. In some embodiments, the method comprises administering a recombinant adeno-associated virus (rAAV, for example a rAAV6) vector to the subject only if the subject does not comprise anti-AAV (e.g. anti-AAV6) antibodies. Any rAAV vector may be administered according to the methods disclosed herein. In some embodiments, the rAAV vector is selected from the group consisting of a rAAV1 vector, a rAAV2 vector, a rAAV3B vector, a rAAV3 vector, a rAAV4 vector, a rAAV5 vector, a rAAV6 vector, a rAAV7 vector, a rAAV8 vector, a rAAV9 vector, a rAAV10 vector, a rAAV11 vector, a rAAVrh10 vector, a rAAVrh39 vector, and a rAAVrh74 vector. In some embodiments, the rAAV vector is a rAAV6 vector. In some embodiments, the anti-AAV antibody is selected from the group consisting of an anti-AAV1 antibody, an anti-AAV2 antibody, an anti-AAV3B antibody, an anti-AAV3 antibody, an anti-AAV4 antibody, an anti-AAV5 antibody, an anti-AAV6 antibody, an anti-AAV7 antibody, an anti-AAV8 antibody, an anti-AAV9 antibody, an anti-AAV10 antibody, an anti-AAV11 antibody, an anti-AAVrh10 antibody, an anti-AAVrh39 antibody, and an anti-AAVrh74 antibody. In some embodiments, the anti-AAV antibody is an anti-AAV6 antibody. The skilled artisan would recognize that the subject should be negative for the anti-AAV antibody corresponding to the rAAV vector being administered.

In some embodiments, any anti-AAV (e.g. anti-AAV6) antibodies in the subject are detected using any of methods of detecting anti-AAV (e.g. anti-AAV6) antibodies using a total antibody assay (e.g. a screening assay or a confirmatory assay) disclosed herein. In some embodiments, any anti-AAV (e.g. anti-AAV6) antibodies in the subject are detected using any of methods of detecting anti-AAV (e.g. anti-AAV6) antibodies using a screening assay disclosed herein. In such embodiments, the rAAV (e.g. rAAV6) vector is only administered to the subject of the screening assay does not detect anti-AAV (e.g. anti-AAV6) antibodies in the subject (e.g., the normalized response of the screening is below the cut point factor).

In some embodiments, any anti-AAV (e.g. anti-AAV6) antibodies in the subject are detected using any of methods of detecting anti-AAV (e.g. anti-AAV6) antibodies using a confirmatory assay disclosed herein. In such embodiments, the rAAV (e.g. rAAV6) vector is only administered to the subject of the confirmatory assay does not detect anti-AAV (e.g. anti-AAV6) antibodies in the subject (i.e., the percent inhibition in the confirmatory assay is below the cut point percentage).

In some embodiments, the subject is suffering from a genetic disorder. In some embodiments, the subject is suffering from a neurological disorder. In some embodiments, the subject is administered a rAAV (e.g. rAAV6) vector. In some embodiments, the genetic disorder is a lysosomal storage disease. In some embodiments, the disorder is selected from the group consisting of hemophilia A and B, Fabry disease, sickle cell disease, beta thalassemia, mucopolysaccharidosis type I and II, phenylketonuria, glycogen storage disease type 1a, GLUT1 deficiency syndrome, and HIV/AIDS. In some embodiments, the subject is suffering from hemophilia A. In some embodiments, the subject is suffering from hemophilia B. In some embodiments, the subject is suffering from sickle cell disease. In some embodiments, the subject is suffering from beta thalassemia. In some embodiments, the subject is suffering from mucopolysaccharidosis type I. In some embodiments, the subject is suffering from mucopolysaccharidosis type II. In some embodiments, the subject is suffering from phenylketonuria. In some embodiments, the subject is suffering from glycogen storage disease type 1a. In some embodiments, the subject is suffering from GLUT1 deficiency syndrome. In some embodiments, the subject is suffering from HIV/AIDS. In some embodiments, the subject is suffering from Fabry disease.

In some embodiments, the subject is a mammal. In some embodiments, the subject is a human or a non-human primate. In some embodiments, the subject is a human. In some embodiments, the subject is a non-human primate. In some embodiments, the subject is a monkey. In some embodiments, the subject is a lab animal. In some embodiments, the subject is a pig. In some embodiments, the subject is a rat. In some embodiments, the subject is a mouse. In some embodiments, the subject is a cat. In some embodiments, the subject is a dog. In some embodiments, the subject is a guinea pig. In some embodiments, the subject has not previously received an AAV (e.g. AAV6) vector.

### 6. Numbered Embodiments

Particular embodiments of the disclosure are set forth in the following numbered paragraphs:
A1. A method of detecting anti-adeno-associated virus (anti-AAV) antibodies in a test subject, the method comprising
(a) providing a test sample obtained from the test subject;
(b) contacting the test sample with a test AAV capsid, wherein the test AAV capsid has been immobilized on a test solid support;
(c) contacting the test sample-contacted immobilized test AAV capsid with a test secondary antibody, wherein the test secondary antibody comprises a test detectable label; and
(d) detecting the test detectable label;
wherein if the label detected with a normalized response relative to a negative control is greater than or equal to a cut point factor, then the test subject comprises anti-AAV antibodies; and wherein if the label is detected with a normalized response relative to a negative control is less than the cut point factor, then the test subject does not comprise anti-AAV antibodies.
A2. The method of embodiment A1, wherein the cut point factor is determined by a method comprising:
(1) providing reference samples obtained from a plurality of reference subjects, wherein each of the reference subjects was negative for a neutralizing anti-AAV antibody assay;
(2) contacting each reference sample with a reference AAV capsid, wherein the reference AAV capsid has been immobilized on a reference solid support;
(3) contacting each reference sample-contacted immobilized reference AAV capsid with a reference secondary antibody, wherein the reference secondary antibody comprises a reference detectable label;
(4) detecting the reference detectable label for each reference sample;
(5) removing outlier reference samples; and
(6) using the outlier-removed reference samples to determine the cut point factor.
A3. The method of embodiment A2, wherein the outlier reference samples are removed using the criteria [Q1−1.5*(Q3−Q1),Q3+1.5*(Q3−Q1)].
A4. The method of embodiment A2 or A3, wherein the cut point factor is based on a parametric method with a 95% confidence interval.
A5. The method of embodiment A2 or A3, wherein the cut point factor is based on a robust parametric method with a 95% confidence interval.
A6. The method of embodiment A2 or A3, wherein the cut point factor is based on a non-parametric method.
A7. The method of any one of embodiments A1-A6, wherein the anti-AAV antibodies are anti-AAV6 antibodies and the AAV capsid is an AAV6 capsid.
A8. The method of any one of embodiments A1-A7, wherein the test sample comprises a fluid selected from the group consisting of serum, plasma, saliva, cerebral spinal fluid, and ocular fluid.
A9. The method of any one of embodiments A1-A8, wherein each reference sample comprises a fluid selected from the group consisting of serum, plasma, saliva, cerebral spinal fluid, and ocular fluid.
A10. The method of any one of embodiments A1-A9, wherein the method further comprises the step of contacting the immobilized AAV capsid with a wash buffer after step (b) and before step (c).
A11. The method of any one of embodiments A1-A10, wherein the method further comprises the step of contacting the sample-contacted immobilized AAV capsid with a wash buffer after step (c) and before step (d).
A12. The method of any one of embodiments A1-A11, wherein the test detectable label and the reference detectable label are independently selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
A13. The method of any one of embodiments A1-A12, wherein the test subject is a human.
A14. The method of any one of embodiments A1-A13, wherein an AAV vector was administered to the test subject before the sample was obtained from the test subject.
A15. A method of detecting anti-adeno-associated virus (anti-AAV) antibodies in a test subject, the method comprising
(a) providing a first test sample and a second test sample obtained from the test subject;
(b) contacting the second test sample with a test soluble AAV capsid;
(c) contacting the first test sample with a first test immobilized AAV capsid, wherein the first test immobilized AAV capsid has been immobilized on a first test solid support;
(d) contacting the mixture of the second test sample and the soluble test AAV capsid with a second test immobilized AAV capsid, wherein the second test immobilized AAV capsid has been immobilized on a second test solid support;
(e) contacting the first test sample-contacted first test immobilized AAV capsid with a first test secondary antibody, wherein the first test secondary antibody comprises a first test detectable label;
(f) contacting the mixture-contacted second test immobilized AAV capsid with a second test secondary antibody, wherein the second test secondary antibody comprises a second test detectable label, wherein the second test secondary antibody is the same as the first secondary antibody and the first test detectable label is the same as the second test detectable label; and
(g) detecting the first and second test detectable labels;
wherein if the amount of second label detected is reduced by a cut point percentage or more compared to the amount of first label detected, then the subject comprises anti-AAV antibodies; and wherein if the amount of second label detected is reduced by less than the cut point compared to the amount of first label detected, then the subject does not comprise anti-AAV antibodies.
A16. The method of embodiment A15 wherein the cut point is determined by a method comprising:
(1) providing first and second reference samples obtained from a plurality of reference subjects, wherein each of the reference subjects was negative for a neutralizing anti-AAV antibody assay;
(2) contacting, for each reference subject, the second reference sample with a reference soluble AAV capsid;
(3) contacting, for each reference subject, the first reference sample with a first reference immobilized AAV capsid, wherein the first reference immobilized AAV capsid has been immobilized on a first reference solid support;
(4) contacting, for each reference subject, the mixture of the second reference sample and the reference soluble AAV capsid with a second reference immobilized AAV capsid, wherein the second reference immobilized AAV capsid has been immobilized on a second reference solid support;
(5) contacting, for each reference subject, the first reference sample-contacted first immobilized AAV capsid with a first secondary antibody, wherein the first secondary antibody comprises a first reference detectable label;
(6) contacting, for each reference subject, the mixture-contacted second immobilized AAV capsid with a second secondary antibody, wherein the second secondary antibody comprises a second reference detectable label, wherein the second secondary antibody is the same as the first secondary antibody and the first reference detectable label is the same as the second reference detectable label; and
(7) detecting, for each reference subject, the first and second reference detectable labels;
(8) removing outlier reference samples; and
(9) using the outlier-removed reference samples to determine the cut point percentage.
A17. The method of embodiment A16, wherein the outlier reference samples are removed using the criteria [Q1−1.5*(Q3−Q1),Q3+1.5*(Q3−Q1)].
A18. The method of embodiment A16 or A17, wherein the cut point percentage is based on a parametric method with a 99% confidence interval.
A19. The method of embodiment A16 or A17, wherein the cut point percentage is based on a robust parametric method with a 99% confidence interval.
A20. The method of embodiment A16 or A17, wherein the cut point percentage is based on a non-parametric method.
A21. The method of any one of embodiments A15-A20, wherein the anti-AAV antibodies are anti-AAV6 antibodies, the soluble AAV capsid is a soluble AAV6 capsid, the first immobilized AAV capsid is a first immobilized AAV6 capsid, and the second immobilized AAV capsid is a second immobilized AAV6 capsid.
A22. The method of any one of embodiments A15-A21, wherein the first and second test sample each comprises a fluid selected from the group consisting of serum, plasma, saliva, cerebral spinal fluid, and ocular fluid.
A23. The method of any one of embodiments A15-A22, wherein each first and second reference sample comprises a fluid selected from the group consisting of serum, plasma, saliva, cerebral spinal fluid, and ocular fluid.
A24. The method of any one of embodiments A15-A23, wherein the method further comprises the step of contacting the first test immobilized AAV6 capsid with a wash buffer after step (c) and before step (e).
A25. The method of any one of embodiments A15-A24, wherein the method further comprises the step of contacting the second test immobilized AAV6 capsid with a wash buffer after step (d) and before step (f).
A26. The method of any one of embodiments A15-A25, wherein the method further comprises the step of contacting the sample-contacted first test immobilized AAV6 capsid with a wash buffer after step (e) and before step (g).
A27. The method of any one of embodiments A15-A26, wherein the method further comprises the step of contacting the mixture-contacted second immobilized AAV6 capsid with a wash buffer after step (f) and before step (g).
A28. The method of any one of embodiments A15-A27, wherein the first and second test detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof
A29. The method of any one of embodiments A15-A28, wherein the first and second reference detectable label are selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
A30. The method of any one of embodiments A15-A29, wherein the test subject is a human.
A31. The method of any one of embodiments A15-A30, wherein an AAV vector was administered to the test subject before the sample was obtained from the test subject.
A32. The method of any one of embodiment A15-A31, wherein the test soluble AAV capsid is at a concentration of 7.5E10 to 15E10 cp/mL.
A33. A method of treating a test subject in need of gene therapy, wherein the method comprises administering a recombinant adeno-associated virus (rAAV) vector to the test subject only if the test subject does not comprise anti-AAV antibodies, wherein any anti-AAV antibodies in the test subject are detected by a method according to any one of embodiments A1-A32.
A34. The method of embodiment A33, wherein the test subject is suffering from a genetic disorder.
A35. The method of embodiment A33, wherein the test subject is suffering from a neurological disorder.
A36. The method of embodiment A34 or A35, wherein the disorder is selected from the group consisting of hemophilia A and B, Fabry disease, sickle cell disease, beta thalassemia, mucopolysaccharidosis type I and II, phenylketonuria, glycogen storage disease type 1a, GLUT1 deficiency syndrome, and HIV/AIDS.
A37. The method of embodiment A36, wherein the disorder is Fabry disease.
A38. The method of any one of embodiments A33-A37, wherein the test subject had not previously been administered an AAV6 vector.
B1. A method of detecting anti-adeno-associated virus (anti-AAV; e.g. anti-AAV6) antibodies in a subject, the method comprising
(a) providing a sample obtained from the subject;
(b) contacting the sample with an AAV (e.g. rAAV6) capsid, wherein the AAV (e.g. rAAV6) capsid has been immobilized on a solid support;
(c) contacting the sample-contacted immobilized AAV (e.g. rAAV6) capsid with a secondary antibody, wherein the secondary antibody comprises a detectable label; and
(d) detecting the label;
wherein if the label detected with a normalized response relative to a negative control is1.5 or greater, then the subject comprises anti-AAV (e.g. anti-AAV6) antibodies; and wherein if the label is detected with a normalized response relative to a negative control is less than 1.5, then the subject does not comprise anti-AAV (e.g. anti-AAV6) antibodies.
B2. The method of embodiment B1, wherein if the normalized response relative to the negative control is 1.6 or greater, then the subject comprises anti-AAV (e.g. anti-AAV6) antibodies; and wherein if the normalized response relative to the negative control is less than 1.6, then the subject does not comprise anti-AAV (e.g. anti-AAV6) antibodies.
B3. The method of embodiment B1, wherein if the normalized response relative to the negative control is 1.7 or greater, then the subject comprises anti-AAV (e.g. anti-AAV6) antibodies; and wherein if the normalized response relative to the negative control is less than 1.7, then the subject does not comprise anti-AAV (e.g. anti-AAV6) antibodies.
B4. The method of embodiment B1, wherein if the normalized response relative to the negative control is 1.73 or greater, then the subject comprises anti-AAV (e.g. anti-AAV6) antibodies; and wherein if the normalized response relative to the negative control is less than 1.73, then the subject does not comprise anti-AAV (e.g. anti-AAV6) antibodies.
B5. The method of embodiment B1, wherein if the normalized response relative to the negative control is 1.75 or greater, then the subject comprises anti-AAV (e.g. anti-AAV6) antibodies; and wherein if the normalized response relative to the negative control is less than 1.75, then the subject does not comprise anti-AAV (e.g. anti-AAV6) antibodies.
B6. The method of any one of embodiments B1-B5, wherein the sample comprises a fluid selected from the group consisting of serum, plasma, saliva, cerebral spinal fluid, and ocular fluid.
B7. The method of any one of embodiments B1-B6, wherein the method further comprises the step of contacting the immobilized AAV (e.g. rAAV6) capsid with a wash buffer after step (b) and before step (c).
B8. The method of embodiment B7, wherein the wash buffer comprises 1× phosphate buffered saline, 0.05% Tween 20.
B9. The method of any one of embodiments B1-B8, wherein the method further comprises the step of contacting the sample-contacted immobilized AAV (e.g. rAAV6) capsid with a wash buffer after step (c) and before step (d).
B10. The method of embodiment B9, wherein the wash buffer comprises 1× phosphate buffered saline, 0.05% Tween 20.
B11. The method of any one of embodiments B1-B10, wherein the detectable label is selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
B12. The method of embodiment B11, wherein the detectable label is a colorimetric label.
B13. The method of embodiment B12, wherein the detectable label is horseradish peroxidase.
B14. The method of any one of embodiments B1-B13, wherein the solid support is a well of a culture plate.
B15. The method of any one of embodiments B1-B14, wherein the subject is a mammal.
B16. The method of embodiment B15, wherein the subject is a human.
B17. The method of embodiment B16, wherein the secondary antibody is an anti-human IgG antibody.
B18. The method of any one of embodiments B1-B17, wherein an AAV (e.g. rAAV6) vector was administered to the subject before the sample was obtained from the subject.
B19. The method of embodiment B1-B18, wherein the negative control is a prescreened negative sample.
B20. The method of embodiment B19, wherein the negative control was prescreened in a neutralizing antibody assay.
B21. A method of detecting anti-adeno-associated virus (anti-AAV; e.g. anti-AAV6) antibodies in a subject, the method comprising
(a) providing a first sample and a second sample obtained from the subject;
(b) contacting the second sample with a soluble AAV (e.g. rAAV6) capsid;
(c) contacting the first sample with a first immobilized AAV (e.g. rAAV6) capsid, wherein the first immobilized AAV (e.g. rAAV6) capsid has been immobilized on a first solid support;
(d) contacting the mixture of the second sample and the soluble AAV (e.g. rAAV6) capsid with a second immobilized AAV (e.g. rAAV6) capsid, wherein the second immobilized AAV (e.g. rAAV6) capsid has been immobilized on a second solid support;
(e) contacting the first sample-contacted first immobilized AAV (e.g. rAAV6) capsid with a first secondary antibody, wherein the first secondary antibody comprises a first detectable label;
(f) contacting the mixture-contacted second immobilized AAV (e.g. rAAV6) capsid with a second secondary antibody, wherein the second secondary antibody comprises a second detectable label, wherein the second secondary antibody is the same as the first secondary antibody and the second detectable label is the same as the second detectable label; and
(g) detecting the first and second labels;
wherein if the amount of second label detected is reduced by 20% or more compared to the amount of first label detected, then the subject comprises anti-AAV (e.g. anti-AAV6) antibodies; and wherein if the amount of second label detected is reduced by less than 20% compared to the amount of first label detected, then the subject does not comprise anti-AAV (e.g. anti-AAV6) antibodies.
B22. The method of embodiment B21 wherein, if the amount of second label detected is reduced by 22% or more compared to the amount of first label detected, then the subject comprises anti-AAV (e.g. anti-AAV6) antibodies; and wherein if the amount of second label detected is reduced by less than 22% compared to the amount of first label detected, then the subject does not comprise anti-AAV (e.g. anti-AAV6) antibodies.
B23. The method of embodiment B21, wherein if the amount of second label detected is reduced by 24% or more compared to the amount of first label detected, then the subject comprises anti-AAV (e.g. anti-AAV6) antibodies; and wherein if the amount of second label detected is reduced by less than 24% compared to the amount of first label detected, then the subject does not comprise anti-AAV (e.g. anti-AAV6) antibodies.
B24. The method of embodiment B21, wherein if the amount of second label detected is reduced by 24.1% or more compared to the amount of first label detected, then the subject comprises anti-AAV (e.g. anti-AAV6) antibodies; and wherein if the amount of second label detected is reduced by less than 24.1% compared to the amount of first label detected, then the subject does not comprise anti-AAV (e.g. anti-AAV6) antibodies.
B25. The method of embodiment B21, wherein if the amount of second label detected is reduced by 24.5% or more compared to the amount of first label detected, then the subject comprises anti-AAV (e.g. anti-AAV6) antibodies; and wherein if the amount of second label detected is reduced by less than 24.5% compared to the amount of first label detected, then the subject does not comprise anti-AAV (e.g. anti-AAV6) antibodies.
B26. The method of any one of embodiments B21-B25, wherein the first and second sample each comprises a fluid selected from the group consisting of serum, plasma, saliva, cerebral spinal fluid, and ocular fluid.
B27. The method of any one of embodiments B21-B26, wherein the method further comprises the step of contacting the first immobilized AAV (e.g. rAAV6) capsid with a wash buffer after step (c) and before step (e).
B28. The method of any one of embodiments B21-B27, wherein the method further comprises the step of contacting the second immobilized AAV (e.g. rAAV6) capsid with a wash buffer after step (d) and before step (f).
B29. The method of embodiment B27 or B28, wherein the wash buffer comprises of 1× phosphate buffered saline, 0.05% Tween 20.
B30. The method of any one of embodiments B21-B29, wherein the method further comprises the step of contacting the sample-contacted first immobilized AAV (e.g. rAAV6) capsid with a wash buffer after step (e) and before step (g).
B31. The method of any one of embodiments B21-B30, wherein the method further comprises the step of contacting the mixture-contacted second immobilized AAV (e.g. rAAV6) capsid with a wash buffer after step (f) and before step (g).
B32. The method of embodiment B30 or B31, wherein the wash buffer comprises of 1× phosphate buffered saline, 0.05% Tween 20.
B33. The method of any one of embodiments B21-B32, wherein the first and second detectable label are each selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
B34. The method of embodiment B33, wherein the first and second detectable labels are each a colorimetric label.
B35. The method of embodiment B34, wherein the first and second detectable labels are each horseradish peroxidase.
B36. The method of any one of embodiments B21-B35, wherein the first and second solid support are each a well of a culture plate.
B37. The method of any one of embodiments B21-B36, wherein the subject is a mammal.
B38. The method of embodiment B37, wherein the subject is a human.
B39. The method of embodiment B38, wherein the secondary antibody is an anti-human IgG antibody.
B40. The method of any one of embodiments B21-B39, wherein an AAV (e.g. rAAV6) vector was administered to the subject before the sample was obtained from the sample.
B41. The method of any one of embodiment B21-B40, wherein the soluble AAV (e.g. rAAV6) capsid is at a concentration of 7.5E10 to 15E10 cp/mL.
B42. A method of treating a subject in need of gene therapy, wherein the method comprises administering a recombinant adeno-associated virus (rAAV, e.g. rAAV6) vector to the subject only if the subject does not comprise anti-AAV (e.g. anti-AAV6) antibodies, wherein any anti-AAV (e.g. anti-AAV6) antibodies in the subject are detected by a method comprising:
(a) providing a sample obtained from the subject;
(b) contacting the sample with an AAV (e.g. rAAV6) capsid, wherein the AAV (e.g. rAAV6) capsid has been immobilized on a solid support;
(c) contacting the sample-contacted immobilized AAV (e.g. rAAV6) capsid with a secondary antibody, wherein the secondary antibody comprises a detectable label; and
(d) detecting the label;
wherein if the label detected with a normalized response relative to a negative control is 1.5 or greater, then the subject comprises anti-AAV (e.g. anti-AAV6) antibodies; and wherein if the label detected with a normalized response relative to a negative control is less than 1.5, then the subject does not comprise anti-AAV (e.g. anti-AAV6) antibodies.
B43. The method of embodiment B42, wherein if the normalized response relative to the negative control is 1.6 or greater, then the subject comprises anti-AAV (e.g. anti-AAV6) antibodies; and wherein if the normalized response relative to the negative control is less than 1.6, then the subject does not comprise anti-AAV (e.g. anti-AAV6) antibodies.
B44. The method of embodiment B42, wherein if the normalized response relative to the negative control is 1.7 or greater, then the subject comprises anti-AAV (e.g. anti-AAV6) antibodies; and wherein if the normalized response relative to the negative control is less than 1.7, then the subject does not comprises anti-AAV (e.g. anti-AAV6) antibodies.
B45. The method of embodiment B42, wherein if the normalized response relative to the negative control is 1.73 or greater, then the subject comprises anti-AAV (e.g. anti-AAV6) antibodies; and wherein if the normalized response relative to the negative control is less than 1.73, then the subject does not comprise anti-AAV (e.g. anti-AAV6) antibodies.
B46. The method of embodiment B42, wherein if the normalized response relative to the negative control is 1.75 or greater, then the subject comprises anti-AAV (e.g. anti-AAV6) antibodies; and wherein if the normalized response relative to the negative control is less than 1.75, then the subject does not comprise anti-AAV (e.g. anti-AAV6) antibodies.
B47. The method of any one of embodiments B42-B46, wherein the sample comprises a fluid selected from the group consisting of serum, plasma, saliva, cerebral spinal fluid, and ocular fluid.
B48. The method of any one of embodiments B42-B47, wherein the method further comprises the step of contacting the immobilized AAV (e.g. rAAV6) capsid with a wash buffer after step (b) and before step (c).
B49. The method of embodiment B48, wherein the wash buffer comprises 1× phosphate buffered saline, 0.05% Tween 20.
B50. The method of any one of embodiments B42-B49, wherein the method further comprises the step of contacting the sample-contacted immobilized AAV (e.g. rAAV6) capsid with a wash buffer after step (c) and before step (d).
B51. The method of embodiment B50, wherein the wash buffer comprises of 1× phosphate buffered saline, 0.05% Tween 20.
B52. The method of any one of embodiments B42-B51, wherein the detectable label is selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
B53. The method of embodiment B52, wherein the detectable label is a colorimetric label.
B54. The method of embodiment B53, wherein the detectable label is horseradish peroxidase.
B55. The method of any one of embodiments B42-B54, wherein the solid support is a well of a culture plate.
B56. The method of any one of embodiments B42-B55, wherein the subject is a mammal.
B57. The method of embodiment B56, wherein the subject is a human.
B58. The method of embodiment B57, wherein the secondary antibody is an anti-human IgG antibody.
B59. The method of embodiment B42-B58, wherein the negative control is a predetermined negative sample.
B60. A method of treating a subject in need of gene therapy, wherein the method comprises administering a recombinant adeno-associated virus (rAAV, e.g., rAAV6) vector to the subject only if the subject does not comprise anti-AAV (e.g. anti-AAV6) antibodies, wherein any anti-AAV (e.g. anti-AAV6) antibodies in the subject are detected by a method comprising
(a) providing a first sample and a second sample obtained from the subject;
(b) contacting the second sample with a soluble AAV (e.g. rAAV6) capsid;
(c) contacting the first sample with a first immobilized AAV (e.g. rAAV6) capsid, wherein the first immobilized AAV (e.g. rAAV6) capsid has been immobilized on a first solid support;
(d) contacting the mixture of the second sample and the soluble AAV (e.g. rAAV6) capsid with a second immobilized AAV (e.g. rAAV6) capsid, wherein the second immobilized AAV (e.g. rAAV6) capsid has been immobilized on a second solid support;
(e) contacting the first sample-contacted first immobilized AAV (e.g. rAAV6) capsid with a first secondary antibody, wherein the first secondary antibody comprises a first detectable label;
(f) contacting the mixture-contacted second immobilized AAV (e.g. rAAV6) capsid with a second secondary antibody, wherein the second secondary antibody comprises a second detectable label, wherein the second secondary antibody is the same as the first secondary antibody and the second detectable label is the same as the second detectable label; and
(g) detecting the first and second labels;
wherein if the amount of second label detected is reduced by 20% or more compared to the amount of first label detected, then the subject comprises anti-AAV (e.g. anti-AAV6) antibodies; and wherein if the amount of second label detected is reduced by less than 20% compared to the amount of first label detected, then the subject does not comprise anti-AAV (e.g. anti-AAV6) antibodies.
B61. The method of embodiment B60, wherein, if the amount of second label detected is reduced by 22% or more compared to the amount of first label detected, then the subject comprises anti-AAV (e.g. anti-AAV6) antibodies; and wherein if the amount of second label detected is reduced by less than 22% compared to the amount of first label detected, then the subject does not comprise anti-AAV (e.g. anti-AAV6) antibodies.
B62. The method of embodiment B60, wherein if the amount of second label detected is reduced by 24% or more compared to the amount of first label detected, then the subject comprises anti-AAV (e.g. anti-AAV6) antibodies; and wherein if the amount of second label detected is reduced by less than 24% compared to the amount of first label detected, then the subject does not comprise anti-AAV (e.g. anti-AAV6) antibodies.
B63. The method of embodiment B60, wherein if the amount of second label detected is reduced by 24.1% or more compared to the amount of first label detected, then the subject comprises anti-AAV (e.g. anti-AAV6) antibodies; and wherein if the amount of second label detected is reduced by less than 24.1% compared to the amount of first label detected, then the subject does not comprise anti-AAV (e.g. anti-AAV6) antibodies.
B64. The method of embodiment B60, wherein if the amount of second label detected is reduced by 24.5% or more compared to the amount of first label detected, then the subject comprises anti-AAV (e.g. anti-AAV6) antibodies; and wherein if the amount of second label detected is reduced by less than 24.5% compared to the amount of first label detected, then the subject does not comprise anti-AAV (e.g. anti-AAV6) antibodies.
B65. The method of any one of embodiments B60-B64, wherein the first and second sample each comprises a fluid selected from the group consisting of serum, plasma, saliva, cerebral spinal fluid, and ocular fluid.
B66. The method of any one of embodiments B60-B65, wherein the method further comprises the step of contacting the first immobilized AAV (e.g. rAAV6) capsid with a wash buffer after step (c) and before step (e).
B67. The method of any one of embodiments B60-B66, wherein the method further comprises the step of contacting the second immobilized AAV (e.g. rAAV6) capsid with a wash buffer after step (d) and before step (f).
B68. The method of embodiment B66 or B67, wherein the wash buffer comprises of 1× phosphate buffered saline, 0.05% Tween 20.
B69. The method of any one of embodiments B60-B68, wherein the method further comprises the step of contacting the sample-contacted first immobilized AAV (e.g. rAAV6) capsid with a wash buffer after step (e) and before step (g).
B70. The method of any one of embodiments B60-B69, wherein the method further comprises the step of contacting the mixture-contacted second immobilized AAV (e.g. rAAV6) capsid with a wash buffer after step (f) and before step (g).
B71. The method of embodiment B69 or B70, wherein the wash buffer comprises of 1× phosphate buffered saline, 0.05% Tween 20.
B72. The method of any one of embodiments B60-B71, wherein the first and second detectable label are each selected from the group consisting of fluorescent labels, fluorogenic labels, dyes, colorimetric labels, radioactive labels, luminescent labels, chemiluminescent labels, magnetic particles, metal particles, charged particles, ionic solutions, spores and enzymatic labels or combinations thereof.
B73. The method of embodiment B72, wherein the first and second detectable labels are each a colorimetric label.
B74. The method of embodiment B73, wherein the first and second detectable labels are each horseradish peroxidase.
B75. The method of any one of embodiments B60-B74, wherein the first and second solid support are each a well of a culture plate.
B76. The method of any one of embodiments B60-B75, wherein the subject is a mammal.
B77. The method of embodiment B76, wherein the subject is a human.
B78. The method of embodiment B77, wherein the secondary antibody is an anti-human IgG antibody.
B79. The method of any one of embodiment B60-B78, wherein the soluble AAV (e.g. rAAV6) capsid is at a concentration of7.5E10 -1.5E11 cp/mL.
B80. The method of any one of embodiments B42-B79, wherein the subject is suffering from a genetic disorder.
B81. The method of any one of embodiments B42-B79, wherein the subject is suffering from a neurological disorder.
B82. The method of embodiment B80 or B81, wherein the disorder is selected from the group consisting of hemophilia A and B, Fabry disease, sickle cell disease, beta thalassemia, mucopolysaccharidosis type I and II, phenylketonuria, glycogen storage disease type 1a, GLUT1 deficiency syndrome, and HIV/AIDS.
B83. The method of embodiment B82, wherein the disorder is Fabry disease.
B84. The method of any one of embodiments B42-B83, wherein the subject had not previously been administered an AAV (e.g. rAAV6) vector.

### EXAMPLES

### Example 1

### Neutralizing Antibody Assay

To further increase sensitivity and selectivity of the assay, negative control (NC) samples were selected using an AAV6 neutralizing antibody (NAb) activity assay. See, e.g., FIG. 1A. Briefly, serial dilutions of sample serum were incubated with AAV6-based reporter vectors, carrying luciferase genes to allow any neutralizing antibodies in the serum to bind the reporter vector. After 1 hour, samples were transferred to a 96 well plate and incubated with HT1080 cells. After 22 hours, all wells were read on a microplate reader to determine luciferase expression. The percentage of neutralization for each serum dilution was calculated after normalized to negative control. Each sample was run in duplicate and each plate included positive controls (wells with no serum but AAV6-luciferase vectors), negative controls (wells with only cell culture media), and negative control sample serum.

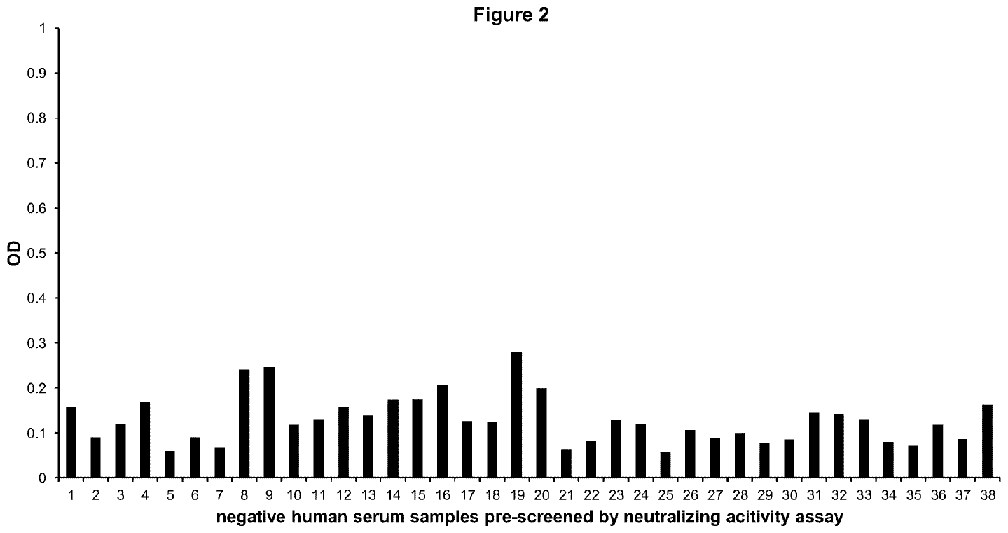

Serum samples with a low concentration in the screening assay (OD=˜0.1) were selected as negative control (NC) samples (FIG. 2).

### Example 2

### Screening Assay for Anti-AAV6 Antibodies for Human Serum

The procedure described herein was used to evaluate the presence of anti-AAV6 total antibodies in human serum. A High Binding Easy Wash Microtiter ELISA plate (Corning, Catalog #: 3369) was coated with 100 μL/well of AAV6 capsid (1.5e10 cp/mL; Sangamo, REC-000841-130178) diluted in 1× Dulbecco's phosphate buffered saline (DPBS), sealed and incubated overnight at 2-8° C. The next day, the plate was incubated at room temperature for a minimum of 15 minutes before plate sealant was removed, and the plate was washed with 300 μL/well wash buffer (1× PBS 0.05% Tween 20) three times. After excess liquid was removed, 200 μL/well of assay diluent (0.5% bovine serum albumin (BSA) in wash buffer) was added to block the plate, and the plate was covered with plate sealer and incubated at room temperature with gentle agitation for 2-3 hours. After removing the plate sealant, the plate was washed with 300 μL/well wash buffer three times and excess liquid removed.

After the plate was blocked for a minimum of 2 hours, 100 μL/well of controls which included negative controls (pooled serum, Sangamo,1650-28), serial dilutions of positive titer control (500 μg/mL of positive source antibody in negative control serum, Sangamo 1732-5), and serum test samples were added to the plate. The plate was covered and incubated at room temperature with gentle agitation for 2 hours±10 minutes. After the plate sealant was removed, the plate was washed with 300 μL/well with wash buffer six times and excess liquid was removed. To detect the anti-AAV6 antibodies, 100 μL/well of diluted anti-human IgG horseradish peroxidase (HRP)-conjugated detection antibody (1:40,000) was added. The plate was then covered with plate sealer and incubated for 1 hour ±5 minutes at room temperature with agitation. The plate was washed with 300 μL/well wash buffer six times and excess liquid was removed. The presence of HRP was detected with an absorbance plate reader using the 3, 3′, 5, 5′-tetramethylbenzidine (TMB) two part kit (KPL, kit #50-76-00, A:5120-0049, B: 5120-0038), where equal parts solution A (TMB peroxidase substrate) and solution B (Peroxidase solution B) were combined and 100 μL/well of the combined solution was added to the plate. The plate was incubated for 15 minutes±2 minutes at room temperature with agitation to allow for color development, and 100 μL/well of stop solution (1M H₃PO₄; Surmodics, Catalog # SLTP-1000-01) was added to end the color reaction. The plate was read at 450 nm and 650 nm wavelengths with the SpectraMax I3X plate reader (Molecular Devices). All samples were normalized to NC as follows:

$$Normalized response = \frac{Mean OD of sample replicates}{Mean OD of negative control replicates}$$

### Example 3

### Confirmatory Assay

To confirm the specificity of the anti-AAV6 antibodies in subject samples, confirmatory assays were performed. Briefly, an ELISA assay as described in Example 2 was performed, except the serum samples were tested in the presence or absence of soluble AAV6 capsids (7.5e10 cp/mL or 1.5e11 cp/mL). Percent inhibition was calculated as follows:

$$\% inhibition = ( 1 - \frac{soluble rAAV6 inhibited sample OD}{uninhibited samples OD} )$$

### Example 4

### Cut Point Determination Using Random Samples

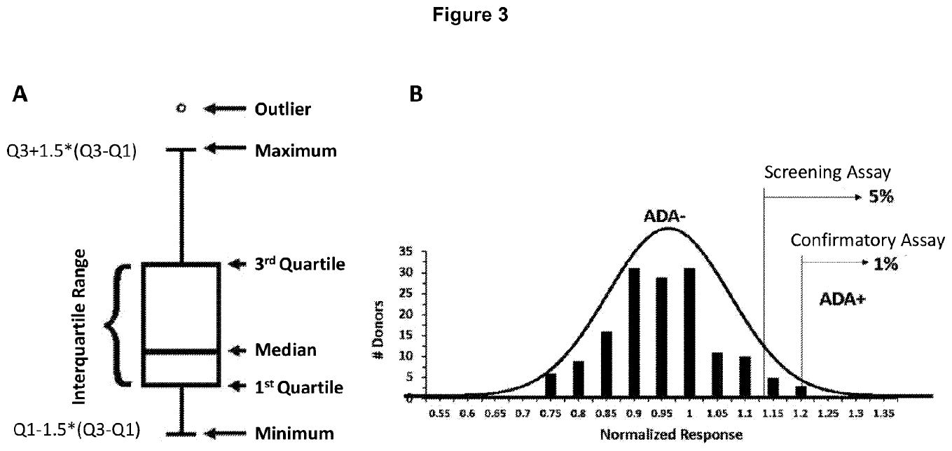

High seroprevalence in healthy donors for anti-AAV6 antibodies does not allow a clear separation between positive and negative cases. Further, screening assay data from these samples are not normally distributed and require several rounds of outlier removals that can affect cut point factor (CPF). The ELISA screening assay of Example 2 and the confirmatory assay of Example 3 were performed using 102 random healthy samples. When screened using the neutralizing antibody assay (CP previously determined to be 0.3, i.e., (30% activity); samples with a NR less than or equal to 0.3 are positive and samples with a NR above 0.3 are negative), 58 samples were negative, and 44 samples were positive. The normalized responses from the ELISA screening assay were graphed in a box and whisker plot using JMP v13 (SAS Institute) to determine outliers based from quantiles (Q) 1 and 3 (FIG. 3A). Outliers were removed using the following criteria:

[Q1−1.5*(Q3−Q1), Q3+1.5*(Q3−Q1)]

Following three rounds of outlier removal using boxplot for screening assay as described above, 70 samples were used in the final cut point calculation. Following log transformation, parametric calculations for cut point factor (CPF) with a 5% false positive rate (FPR) for the ELISA screening assay and a 1% FPR for confirmatory assay were as follows (see, FIG. 3B):

ELISA Screening assay=Mean+1.645×SD(5% FPR))

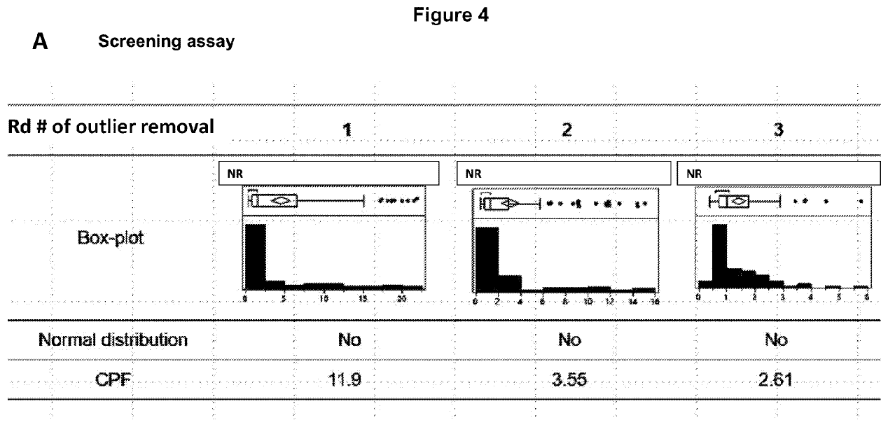

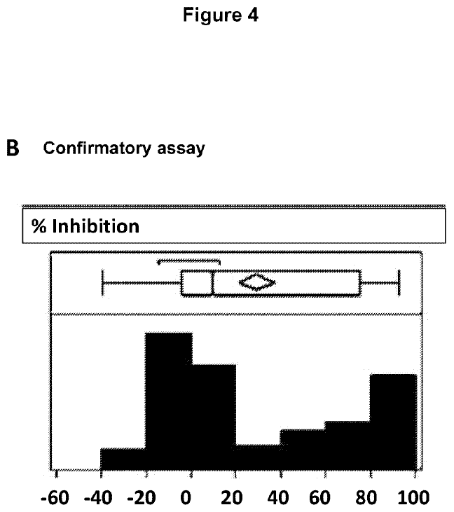

Challenge assay=Mean+2.33×SD (1%FPR)
For random samples, the CPF was determined to be 2.6 for the ELISA screening assay (FIG. 4A) and the CPP was determined to be 43.1% inhibition for the confirmatory assay (FIG. 4B). Samples with values at or above the cut point were positive, and samples with values below the cut point were negative.

### Example 5

### Cut Point Determination Using Prescreened Negative Samples

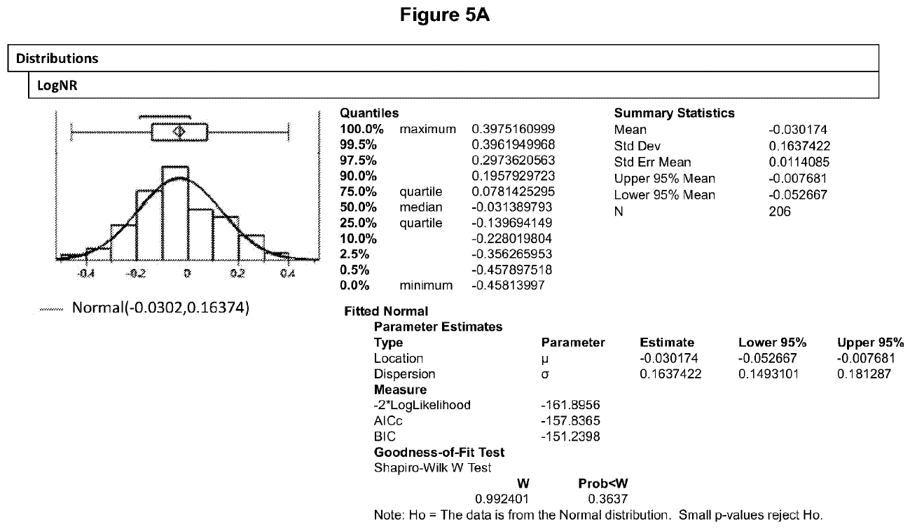

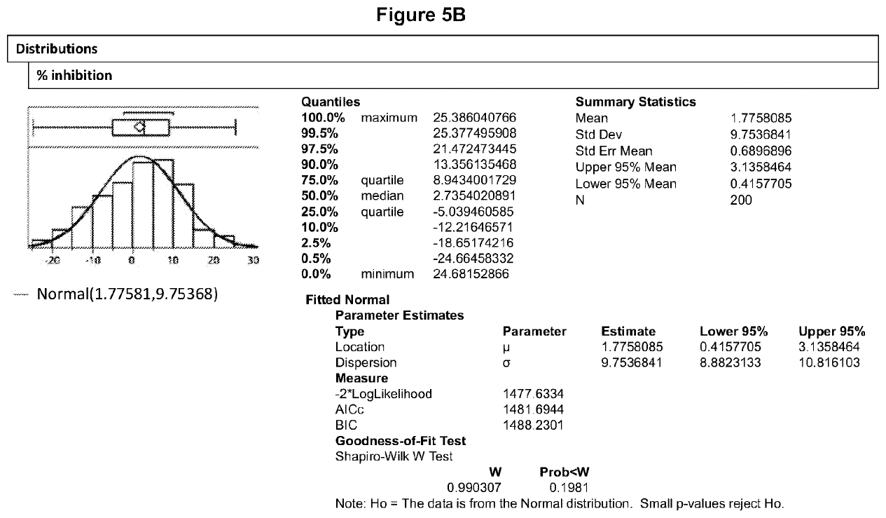

Samples were prescreened using NAb assay (CP=0.3) to identify negative samples. The ELISA screening assay of Example 2 and the confirmatory assay of Example 3 were performed using 104 prescreened negative samples. Outliers were removed using JMP v13 and log transformation was performed as described in Example 4 where one round of outlier removal was done using boxplot for screening assay and one for confirmatory assay; therefore, 99 samples were used in the final CP calculation. Compared to the random samples in Example 4, which utilized three rounds of outlier removal and resulted in the removal of 32 outliers, the use of prescreened samples resulted in a better data set, which utilized only one round of outlier removal and resulted in the removal of 5 outliers. Indeed, even after three rounds of outlier removal from the random samples of Example 4, the distribution was not normal. See FIG. 4A. By contrast, one round of outlier removal with the prescreened samples provided a normal distribution. The CPF and the CPP using the prescreened negative samples were calculated as shown in Example 4. The CPF for the prescreened negative samples in screening assay (5% FPR) was 1.75, and the CPP for the prescreened negative samples in the confirmatory assay (1% FPR) was 24.1% (FIG. 5).

The ELISA screening assay of Example 2 and the confirmatory assay of Example 3 were repeated using an additional 211 prescreened negative samples. In the repeated experiment, the CPF for the prescreened negative samples in screening assay (5% FPR) was 1.73, and the CPP for the prescreened negative samples in the confirmatory assay (1% FPR) was 24.5%.

### Example 6

### Determination of Assay Sensitivity

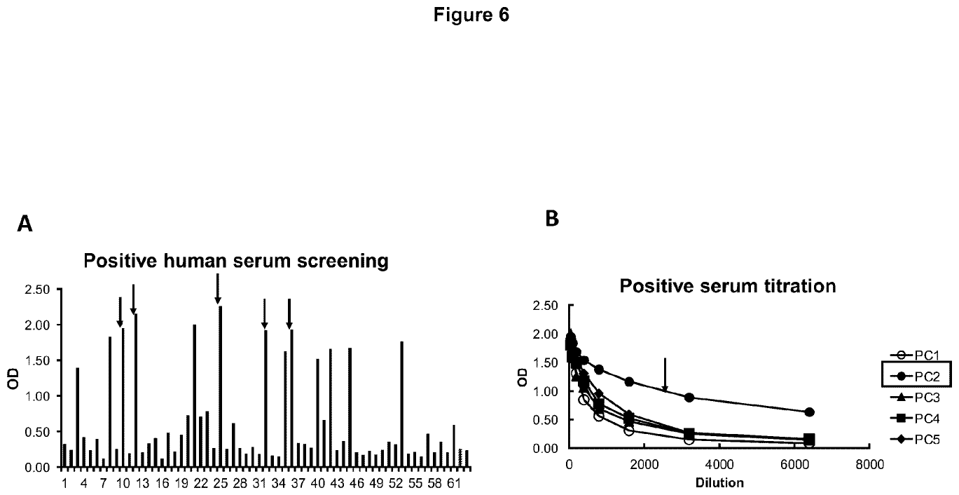

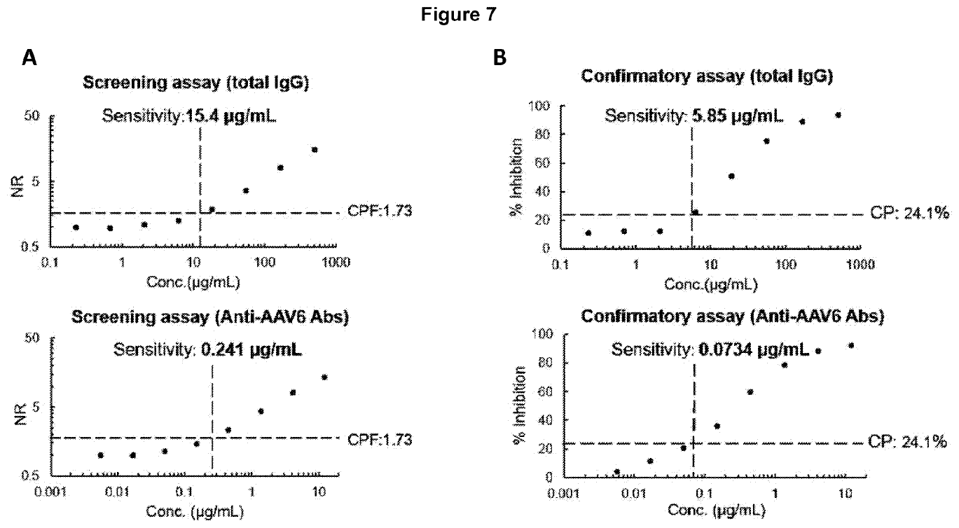

To generate the purified positive control anti-AAV6 antibodies, 63 individual serum samples, which were prescreened as positive for anti-AAV6 antibodies using the neutralizing antibody assay of Example 1, were screened for anti-AAV6 antibodies by ELISA as described in Example 2 (See FIG. 5A and B). Of these 63, the 5 with the highest optical density (OD) were selected, labeled as positive control (PC)-1-5, and titrated (FIG. 6A). The sample with the highest signal across all concentrations on the titration curve, PC-2 (FIG. 6B), was selected for purification of total IgG using a protein A/G spin kit (Thermofisher, 89950) and affinity purification of AAV6 specific antibodies using AAV6-magnetic beads (Pierce™ NHS-activated magnetic beads; Thermofisher, 88827). To assess assay sensitivity, serial dilutions of the total IgG antibody control and affinity purified anti-AAV6 antibodies were assessed using the ELISA screening assay of Example 2 and the confirmatory assay of Example 3 (FIG. 7A). The affinity purified anti-AAV6 antibodies improved sensitivity by more than 64-fold when compared with total IgG antibody control (FIG. 7B). For example, the antibody concentration at the CPF in the ELISA screening assay of Example 2 was 15.4 μg/ml for the total IgG antibody control but only 0.24 μg/ml for the affinity purified anti-AAV6 control. Similarly, the antibody concentration at the CPP in the confirmatory assay of Example 3 was 5.85 μg/ml for the total IgG antibody control but only 0.073 μg/ml for the affinity purified anti-AAV6 control.

### Example 7

### Confirmation of Assay Specificity

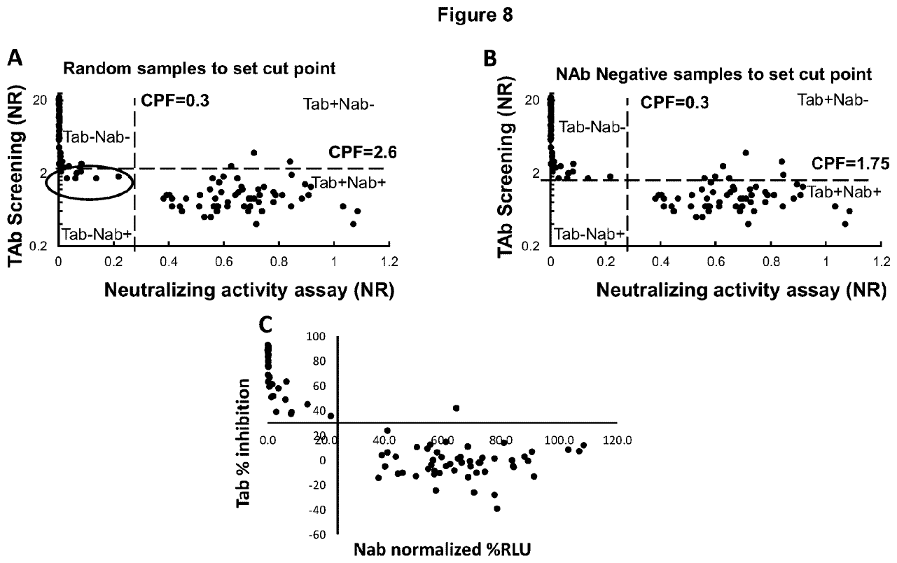

To determine the efficacy of this optimized CPF, 100 samples were evaluated using the neutralizing antibody assay (CP=0.3) of Example 1 and the ELISA screening assay of Example 2. The results from the neutralizing antibody assay (CPF=0.3, i.e., 30% normalized RLU) were compared to the results from the ELISA screening assay using the CPF determined from random healthy samples (CPF=2.6), the results from the ELISA screening assay using the CPF determined using prescreened negative samples (CPF=1.73), and (See, FIG. 8A-8C). When evaluated using CPF=2.6 (random healthy subjects), the ELISA screening assay resulted in 8 false negatives (FIG. 8A, lower left quadrant). Using CPF=1.75 (prescreened negative subjects), the ELISA screening assay results correlated better with the neutralizing antibody assay results, and the false negative results were eliminated (FIG. 8B, left lower quadrant). Similarly, using CPP=24.1% inhibition (prescreened negative subjects), the confirmatory assay results correlated almost perfectly with the neutralizing antibody assay results with no false negative results (FIG. 8C).

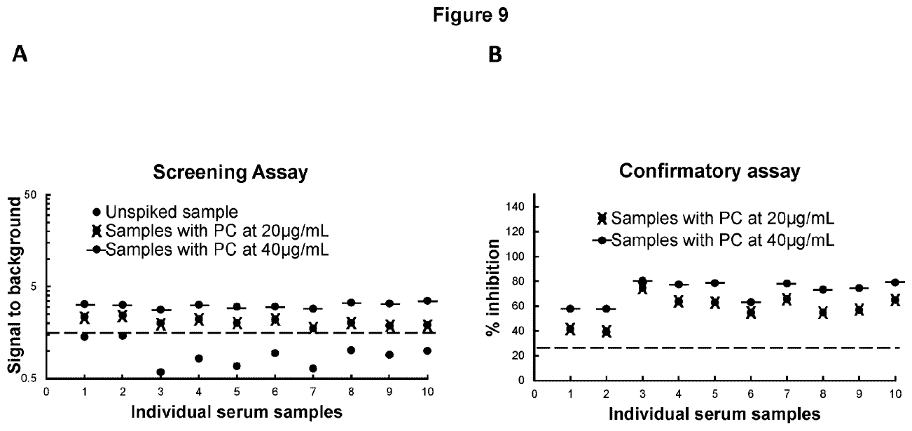

Additionally, the ELISA screening assay of Example 2 and the confirmatory assay of Example 3 were performed using samples that were pre-screened negative for the neutralizing antibody assay of Example 1 in the presence and absence of the positive control antibody purified in Example 6. Specifically, 20 μg/mL or 40 μg/mL of the positive control antibody was added into 10 negative individual serum samples (see FIGS. 9A and 9B). For the ELISA screening assay, using a CPF=1.73, all samples lacking the positive control antibody were negative, and all samples spiked with the positive control antibody were positive (FIG. 9A). Similarly, for the confirmatory assay, using a CP=24.1% inhibition, all samples lacking the positive control were negative, and all samples spiked with the positive control were positive (FIG. 9B). These results confirm the specificity of CPF and CPP, respectively, for the detection of anti-AAV6 antibodies.

The examples set forth above are provided to give those of ordinary skill in the art a complete disclosure and description of how to make and use the embodiments of methods of the invention, and are not intended to limit the scope of what the disclosure. Modifications of the above-described modes for carrying out the disclosure that are obvious to persons of skill in the art are intended to be within the scope of the disclosure. All patents and publications mentioned herein are indicative of the levels of skill of those skilled in the art to which the invention pertains. All references cited herein are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

Many modifications and variations of this disclosure can be made without departing from its spirit and scope, as will be apparent to those skilled in the art. The specific embodiments and examples described herein are offered by way of example only.

1. A method of detecting anti-adeno-associated virus (anti-AAV) antibodies in a test subject, the method comprising
(a) providing a test sample obtained from the test subject;
(b) contacting the test sample with a test AAV capsid, wherein the test AAV capsid has been immobilized on a test solid support;
(c) contacting the test sample-contacted immobilized test AAV capsid with a test secondary antibody, wherein the test secondary antibody comprises a test detectable label; and
(d) detecting the test detectable label;
wherein if the label detected with a normalized response relative to a negative control is greater than or equal to a cut point factor, then the test subject comprises anti-AAV antibodies; and wherein if the label is detected with a normalized response relative to a negative control is less than the cut point factor, then the test subject does not comprise anti-AAV antibodies.

2. The method of claim 1, wherein the cut point factor is determined by a method comprising:
(1) providing reference samples obtained from a plurality of reference subjects, wherein each of the reference subjects was negative for a neutralizing anti-AAV antibody assay;
(2) contacting each reference sample with a reference AAV capsid, wherein the reference AAV capsid has been immobilized on a reference solid support;
(3) contacting each reference sample-contacted immobilized reference AAV capsid with a reference secondary antibody, wherein the reference secondary antibody comprises a reference detectable label;
(4) detecting the reference detectable label for each reference sample;
(5) removing outlier reference samples; and
(6) using the outlier-removed reference samples to determine the cut point factor.

3. The method of claim 2, wherein the outlier reference samples are removed using the criteria [Q1−1.5*(Q3−Q1),Q3+1.5*(Q3−Q1)].

4. The method of claim 2, wherein the cut point factor is based on a parametric method with a 95% confidence interval, a robust parametric method with a 95% confidence interval, or a non-parametric method.

5.-6. (canceled)

7. The method of claim 1, wherein the anti-AAV antibodies are anti-AAV6 antibodies and the AAV capsid is an AAV6 capsid.

8.-9. (canceled)

10. The method of claim 1, wherein the method further comprises:
(1) the step of contacting the immobilized AAV capsid with a wash buffer after step (b) and before step (c); and/or
(2) the step of contacting the sample-contacted immobilized AAV capsid with a wash buffer after step (c) and before step (d).

11.-13. (canceled)

14. The method of claim 1, wherein an AAV vector was administered to the test subject before the sample was obtained from the test subject.

15. A method of detecting anti-adeno-associated virus (anti-AAV) antibodies in a test subject, the method comprising
(a) providing a first test sample and a second test sample obtained from the test subject;
(b) contacting the second test sample with a test soluble AAV capsid;
(c) contacting the first test sample with a first test immobilized AAV capsid, wherein the first test immobilized AAV capsid has been immobilized on a first test solid support;
(d) contacting the mixture of the second test sample and the soluble test AAV capsid with a second test immobilized AAV capsid, wherein the second test immobilized AAV capsid has been immobilized on a second test solid support;
(e) contacting the first test sample-contacted first test immobilized AAV capsid with a first test secondary antibody, wherein the first test secondary antibody comprises a first test detectable label;
(f) contacting the mixture-contacted second test immobilized AAV capsid with a second test secondary antibody, wherein the second test secondary antibody comprises a second test detectable label, wherein the second test secondary antibody is the same as the first secondary antibody and the first test detectable label is the same as the second test detectable label; and
(g) detecting the first and second test detectable labels;
wherein if the amount of second label detected is reduced by a cut point percentage or more compared to the amount of first label detected, then the subject comprises anti-AAV antibodies; and wherein if the amount of second label detected is reduced by less than the cut point compared to the amount of first label detected, then the subject does not comprise anti-AAV antibodies.

16. The method of claim 15 wherein the cut point is determined by a method comprising:
(1) providing first and second reference samples obtained from a plurality of reference subjects, wherein each of the reference subjects was negative for a neutralizing anti-AAV antibody assay;
(2) contacting, for each reference subject, the second reference sample with a reference soluble AAV capsid;
(3) contacting, for each reference subject, the first reference sample with a first reference immobilized AAV capsid, wherein the first reference immobilized AAV capsid has been immobilized on a first reference solid support;
(4) contacting, for each reference subject, the mixture of the second reference sample and the reference soluble AAV capsid with a second reference immobilized AAV capsid, wherein the second reference immobilized AAV capsid has been immobilized on a second reference solid support;
(5) contacting, for each reference subject, the first reference sample-contacted first immobilized AAV capsid with a first secondary antibody, wherein the first secondary antibody comprises a first reference detectable label;
(6) contacting, for each reference subject, the mixture-contacted second immobilized AAV capsid with a second secondary antibody, wherein the second secondary antibody comprises a second reference detectable label, wherein the second secondary antibody is the same as the first secondary antibody and the first reference detectable label is the same as the second reference detectable label;
(7) detecting, for each reference subject, the first and second reference detectable labels;
(8) removing outlier reference samples; and
(9) using the outlier-removed reference samples to determine the cut point percentage.

17. The method of claim 16, wherein the outlier reference samples are removed using the criteria [Q1−1.5*(Q3−Q1),Q3+1.5*(Q3−Q1)].

18. The method of claim 16, wherein the cut point percentage is based on a parametric method with a 99% confidence interval, a robust parametric method with a 95% confidence interval, or a non-parametric method.

19.-20. (canceled)

21. The method of claim 15, wherein the anti-AAV antibodies are anti-AAV6 antibodies, the soluble AAV capsid is a soluble AAV6 capsid, the first immobilized AAV capsid is a first immobilized AAV6 capsid, and the second immobilized AAV capsid is a second immobilized AAV6 capsid.

22.-23. (canceled)

24. The method of claim 15, wherein the method further comprises:
(1) the step of contacting the first test immobilized AAV6 capsid with a wash buffer after step (c) and before step (e);
(2) the step of contacting the second test immobilized AAV6 capsid with a wash buffer after step (d) and before step (f);
(3) the step of contacting the sample-contacted first test immobilized AAV6 capsid with a wash buffer after step (e) and before step (g); and/or
(4) the step of contacting the mixture-contacted second immobilized AAV6 capsid with a wash buffer after step (f) and before step (g).

25.-32. (canceled)

33. A method of treating a test subject in need of gene therapy, wherein the method comprises administering a recombinant adeno-associated virus (rAAV) vector to the test subject only if the test subject does not comprise anti-adeno-associated virus (anti-AAV) antibodies,
wherein any anti-AAV antibodies in the test subject are detected by a method according to claim 15.

34. The method of claim 33, wherein the test subject is suffering from a genetic disorder or a neurological disorder.

35. (canceled)

36. The method of claim 34, wherein the disorder is selected from the group consisting of hemophilia A and B, Fabry disease, sickle cell disease, beta thalassemia, mucopolysaccharidosis type I and II, phenylketonuria, glycogen storage disease type 1a, GLUT1 deficiency syndrome, and HIV/AIDS.

37.-38. (canceled)
